(19) United States
(12) Patent Application Publication
(10) Pub. No.: US 2026/0289696 A1
(43) Pub. Date: Sep. 24, 2026

(54) SYSTEM

(71) Applicant: SoftBank Group Corp., Tokyo (JP)

(72) Inventor: Masayoshi KOIZUMI, Tokyo (JP)

(73) Assignee: SoftBank Group Corp., Tokyo (JP)

(21) Appl. No.: 19/557,242

(22) Filed: Mar. 5, 2026

(30) Foreign Application Priority Data
Mar. 19, 2025 (JP) 2025-044491

(51) Int. Cl. G06Q 50/12; G06N 3/045; G16H 20/60; G16H 50/30

(52) U.S. Cl. CPC G06Q 50/12; G06N 3/045; G16H 20/60; G16H 50/30

(57) ABSTRACT

A system includes a processor that is configured to train a generative artificial intelligence model using personal data, weather forecast data obtained via an internet search tool, recipe data, and shopping product data, generate, by using the generative artificial intelligence model, a prompt for instructing immediate delivery of food optimized for a user on the basis of the personal data, the weather forecast data, the recipe data, and the shopping product data, and generate, by using the generative artificial intelligence model, a prompt for instructing proposal of a meal menu according to an emotion of the user, by recognizing the emotion of the user.

### CROSS-REFERENCE TO RELATED APPLICATION

This application is based on and claims priority under 35 USC 119 from Japanese Patent Application No. 2025-044491 filed on Mar. 19, 2025, the disclosure of which is incorporated by reference herein.

### BACKGROUND

### Technical Field

The Present Disclosure Relates to a System.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional food recommendation and delivery systems handle only limited factors such as simple user preferences or static recipe databases, and therefore cannot provide highly personalized meal proposals that dynamically reflect a user's comprehensive context including real-time weather, health conditions, nutritional requirements, budget, cooking skills, and emotional state. As a result, a user often receives meal suggestions or delivery options that are suboptimal for the user's physical and psychological condition, environmental situation, and practical constraints such as available time and seasonal ingredients. Furthermore, existing systems generally do not utilize a generative artificial intelligence model trained on integrated personal, environmental, and product data to generate prompts that can flexibly instruct downstream services, such as immediate delivery systems or interactive question-answering systems, in natural language. In addition, conventional systems are not configured to recognize a user's emotion and to adapt meal menus accordingly, which limits user satisfaction and adherence to recommended nutrition. There is therefore a need for a system that can train and utilize a generative artificial intelligence model on personal data, weather forecast data, recipe data, and shopping product data, generate prompts that instruct optimal immediate food delivery and emotion-adaptive meal menu proposals, and also generate prompts for presenting optimal recipes and ingredients and for answering various food-related questions.

### SUMMARY

In order to solve the above-described problems, the present invention provides a system comprising a processor, wherein the processor is configured to train a generative artificial intelligence model using personal data, weather forecast data obtained via an internet search tool, recipe data, and shopping product data. The processor is further configured to generate, by using the generative artificial intelligence model, a prompt for instructing immediate delivery of food optimized for a user on the basis of the personal data, the weather forecast data, the recipe data, and the shopping product data. The processor is also configured to recognize an emotion of the user and to generate, by using the generative artificial intelligence model, a prompt for instructing proposal of a meal menu according to the recognized emotion of the user. Moreover, the processor is configured to generate, by using the generative artificial intelligence model, a prompt for instructing presentation of an optimal recipe and ingredients by taking into account at least a budget of the user, a health condition of the user, nutritional aspects, seasonal ingredients, a cooking skill of the user, and a meal preparation time. In addition, the processor is configured to generate, by using the generative artificial intelligence model, a prompt for instructing generation of an answer to a question related to food so as to respond to various questions regarding food. By these means, the system enables highly personalized, context-aware, and emotion-adaptive food delivery, recipe recommendation, and interactive food-related question answering.

The term “processor” refers to one or more hardware processing units, such as a central processing unit (CPU), a graphics processing unit (GPU), or a dedicated accelerator, and may include a combination of such units configured to execute instructions and perform the functions described in the present specification.

The term “personal data” refers to data related to an individual user, including, but not limited to, user preferences, allergies, health conditions, demographic information, dietary restrictions, cooking skill level, budget information, and other profile information used for personalization.

The term “weather forecast data” refers to data representing predicted or current weather conditions for a given geographic location and time, including, but not limited to, temperature, humidity, precipitation, and derived classifications such as “hot,” “cold,” or “mild,” obtained via an internet search tool or weather application programming interface.

The term “internet search tool” refers to an online search service or application programming interface that is capable of retrieving external information, such as weather information or product information, from one or more remote servers over a network.

The term “recipe data” refers to structured or semi-structured information about one or more recipes, including at least a dish name, a list of ingredients, preparation steps, preparation time, difficulty level, nutritional information, and optionally tags such as cuisine type or seasonal suitability.

The term “shopping product data” refers to data describing food products or ingredients available for purchase, including, but not limited to, product names, prices, package sizes, store or vendor information, inventory or availability status, and purchase links or URLs.

The term “generative artificial intelligence model” refers to a machine learning model, such as a neural network, that is trained to generate outputs including natural-language text or other structured data based on input data, and that can be used to generate prompts, recommendations, or answers as described in the present specification.

The term “prompt” refers to a piece of content, typically expressed in natural language or structured text, that is generated by the processor and supplied to a generative artificial intelligence model or downstream system to instruct or guide the generation of an output, such as an instruction for food delivery, a meal proposal, a recipe presentation, or an answer to a question.

The term “immediate delivery of food” refers to an operation or instruction that causes a food delivery service or logistics system to deliver food to a user within a short and practical time frame appropriate for a meal, typically on the same day and preferably within a time window specified or implied by the user or system.

The term “optimized for a user” refers to being determined or selected based on one or more user-specific factors, such as personal data, weather conditions at the user's location, nutritional needs, preferences, budget, and time constraints, so as to increase suitability or satisfaction for that user.

The term “emotion of the user” refers to a psychological or affective state of the user, such as happiness, sadness, stress, fatigue, or excitement, which may be recognized by the system based on user input, behavioral data, biometric data, or other signals.

The term “meal menu” refers to one or more dishes or recipes, possibly including a main dish, side dishes, desserts, and beverages, proposed for consumption in a specific meal, such as breakfast, lunch, or dinner.

The term “budget of the user” refers to a monetary limit or cost preference specified by or inferred for the user, relating to the total allowable or desired expenditure for a meal, ingredients, or ordered food.

The term “health condition of the user” refers to a medical or physiological state of the user, including, but not limited to, chronic diseases, dietary restrictions, allergies, and doctor-recommended limitations, that may affect suitable food or nutritional choices.

The term “nutritional aspects” refers to characteristics of food or recipes related to nutrition, including, but not limited to, caloric content, macronutrient composition (proteins, fats, carbohydrates), micronutrient content (vitamins, minerals), and dietary fiber.

The term “seasonal ingredients” refers to food ingredients that are in season in a given region and time period, typically characterized by better availability, freshness, quality, or price compared to other times of the year.

The term “cooking skill of the user” refers to an estimated or declared level of the user's cooking proficiency, such as beginner, intermediate, or advanced, which may influence the complexity or difficulty level of recipes proposed to the user.

The term “meal preparation time” refers to an estimated amount of time required to prepare and cook a meal or recipe from start to finish, including necessary preparation steps such as washing, cutting, mixing, and cooking.

The term “question related to food” refers to any user query that concerns food, meals, recipes, ingredients, nutrition, cooking methods, food safety, or similar topics addressed by the system.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

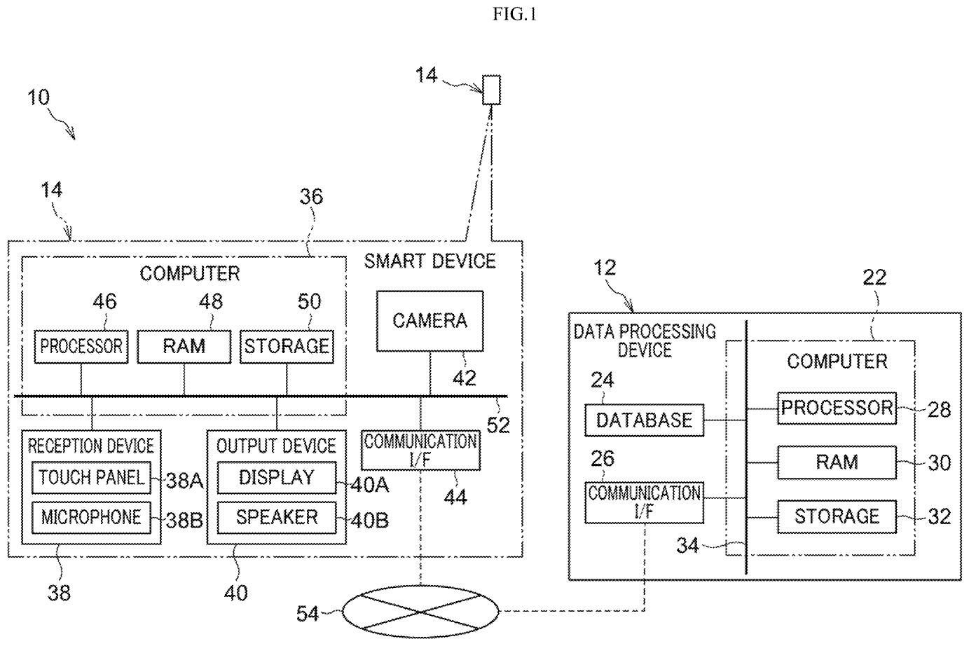

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

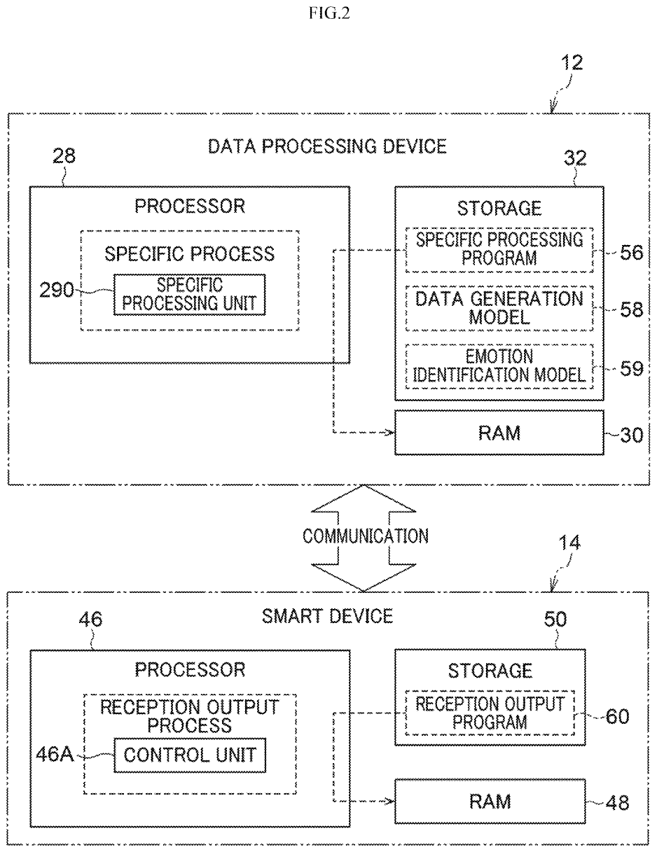

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented meal recommendation systems typically rely on fixed rules, static recipe databases, or simple filtering based on user preferences and allergies. Such systems suffer from multiple technical limitations. First, they often treat heterogeneous data sources, such as user profiles, environmental conditions, and product availability, in isolation. As a result, the underlying processors perform repetitive, fragmented queries and redundant computations across separate subsystems, which increases processing latency, memory usage, and network traffic. Second, existing systems generally do not generate machine-oriented instruction sequences, such as well-structured prompt sentences for a generative AI model, in a way that is dynamically adapted to real-time contextual data, including current weather data, budget constraints, user behavior history, and product availability. This lack of integrated, context-aware prompt generation leads to inefficient use of computational resources of the generative AI model, because the model is either under-constrained, requiring more tokens and post-processing, or over-constrained, requiring repeated invocations to refine results.

Further, conventional systems often require separate application logic to map recommended recipes to purchasable items and to generate user-facing purchase support information, such as store locations and online purchase links. This separation causes duplicated data transformations, additional database lookups, and complex error handling, which collectively degrade system throughput and reliability. Additionally, most existing systems do not feed back user evaluation information or behavior history information into the data preparation and prompt construction pipeline in a structured manner. As a result, the processor cannot effectively optimize subsequent prompt sentences or data integration steps, and cannot reduce unnecessary calls to external services or the generative AI model.

Therefore, there is a need for an improved computer-implemented technique in which a processor systematically integrates personal data, context data, recipe data, and product data, automatically constructs optimized prompt sentences for a generative AI model, and efficiently generates machine-readable purchase support information, while adaptively updating internal data and prompt-generation conditions based on user feedback. Such a technique should improve the functioning of the underlying computing system itself, by reducing redundant data access, lowering processing latency, and improving the efficiency and accuracy of generative AI model invocation and subsequent data handling.

The specific processing by the specific processing unit 290 of the data processing device 12 in Example 1 is realized by the following means.

The present invention provides a server comprising a processor configured to cause a storage unit to store personal data, weather data acquired via an information acquisition unit, cooking information data including cooking procedures, and product information data including sales information, to generate, based on the personal data, the weather data, the cooking information data, and the product information data, a prompt sentence that instructs a generative AI model to generate a meal plan adapted to a preference and a health condition of a user and to a weather condition at a current time, to supply the prompt sentence and associated contextual data to the generative AI model and obtain, from the generative AI model, the meal plan, to associate ingredient information included in the obtained meal plan with the product information data, to specify items purchasable as products that correspond to ingredients required for the meal plan, to generate purchase support information including sales location information and online purchase link information for the specified products, to control a terminal device to display the meal plan and the purchase support information, and to update the personal data and conditions for generating the prompt sentence based on evaluation information or behavior history information acquired from the terminal device so that the updated personal data and the updated conditions are reflected in a subsequent meal plan generation process. This enables the computing system to integrate heterogeneous data sources in a single optimized processing pipeline, to construct and refine machine-oriented prompt sentences that more efficiently guide the generative AI model, to reduce redundant data queries and post-processing operations, and to adapt internal data structures and control parameters based on user feedback, thereby improving processing efficiency, responsiveness, and accuracy of the overall computer-implemented meal recommendation and purchase support functionality.

The term “personal data” refers to data representing attributes of a user, including at least preference information, dietary restrictions, health-related conditions, budget-related information, and historical behavior or usage records, which are stored and processed by the system for generating meal plans.

The term “weather data” refers to data representing environmental conditions of a geographical region at a given time, including at least temperature, humidity, precipitation, and weather condition indicators, which are acquired via an external information acquisition unit and used as context for meal plan generation.

The term “information acquisition unit” refers to a hardware and/or software component configured to obtain data from external data sources via a communication network, including at least application programming interfaces, web services, and data feeds, and to provide the obtained data to a processor or a storage unit.

The term “cooking information data” refers to data representing preparation of food items, including at least recipe identifiers, ingredient lists, cooking procedures, cooking times, and classification tags, which are usable by the system when generating meal plans.

The term “product information data” refers to data representing purchasable items related to food, including at least product identifiers, item names, prices, stock statuses, sales locations, and online purchase links, which are usable by the system for specifying ingredients as purchasable products.

The term “generative AI model” refers to a machine learning model configured to generate output data, including at least natural language text or structured data, in response to input data or instructions, and implemented by software executing on a computing device or on a remote server accessible via a communication network.

The term “prompt sentence” refers to a sequence of machine-readable instructions, expressed in natural language and/or structured text, that encodes constraints, conditions, and context information, and that is supplied to the generative AI model to control generation of a meal plan, answer information, or other output.

The term “meal plan” refers to data representing at least one recommended food combination for a user for a specific time period, including at least dish names, associated ingredient information, and optionally reasons or explanations corresponding to user conditions or environmental conditions.

The term “ingredient information” refers to data representing raw materials or components used to prepare a dish, including at least ingredient names, quantities, and optionally dietary attributes, which are associated with product information data for purchase support.

The term “purchase support information” refers to data generated by the processor that assists a user in acquiring ingredients or products, including at least a mapping between ingredients and purchasable products, sales location information, and online purchase link information.

The term “sales location information” refers to data that identifies a place or service through which a product can be purchased, including at least store names, store identifiers, and optionally geographical location information or distance-related information.

The term “online purchase link information” refers to data that specifies a network resource, including at least a uniform resource locator or equivalent identifier, through which a user can initiate or complete an electronic purchase of a product.

The term “terminal device” refers to a user-operated computing device, including at least a mobile terminal, a tablet terminal, a personal computer, or an equivalent display-capable device, configured to communicate with the server, display information, and accept user input.

The term “evaluation information” refers to data representing explicit or implicit feedback from a user regarding a meal plan or a system output, including at least ratings, selection or rejection of proposed plans, or textual comments, which are used to update personal data or prompt-generation conditions.

The term “behavior history information” refers to data representing actions performed by a user through the terminal device or related services, including at least viewing actions, selection actions, purchase actions, and interaction timestamps, which are recorded and used to adjust subsequent processing.

The term “constraint conditions” refers to data representing limitations or requirements that must be satisfied by the generative AI model output, including at least budget limits, nutritional limits, time limits, dietary restrictions, and environmental constraints.

The term “priority conditions” refers to data representing preferences or weighting factors that guide the generative AI model toward preferred outputs among solutions satisfying the constraint conditions, including at least user taste preferences, preparation simplicity, seasonality, and nutritional emphasis.

The term “answer information” refers to data generated by the generative AI model in response to inquiry information, including at least natural language explanations, recommendations, or instructions relating to food, nutrition, cooking methods, or purchasing methods.

In an embodiment, a server cooperates with one or more terminal devices to provide context-aware meal planning and purchase support based on heterogeneous data sources and a generative AI model. The server executes software modules on general-purpose computing hardware, while each terminal executes a client application and provides user interaction and local sensing capabilities.

The server uses a computing platform such as a rack-mounted or cloud-based computer system including at least one central processing unit (CPU), an optional graphics processing unit (GPU), a volatile memory device (for example, a main memory), a non-volatile storage device (for example, a solid-state drive), and a network interface. The server executes an operating system, such as a general-purpose server operating system, and middleware or frameworks such as a web application framework and a machine learning framework. For example, the server uses an application framework implemented in a high-level language environment and a machine learning framework such as a tensor-based computation library to perform numerical operations for a generative AI model.

The terminal uses a mobile computing device, such as a smartphone or a tablet, including at least a processor, a memory, a display, an input sensor such as a touch panel, a location acquisition component, and a wireless communication interface. The terminal executes a native application or a web-based application capable of communicating with the server via a network using a standardized communication protocol.

The server stores personal data representing a user profile, weather data acquired via an information acquisition unit from an external weather information service, cooking information data including recipe identifiers, ingredient lists, and cooking procedures, and product information data including product identifiers, prices, stock statuses, and sales locations in a structured data store such as a relational database system or a key-value store. The server represents each category of data using predetermined data structures. For example, the server maintains a user profile table including fields for preference vectors, allergy flags, health-condition labels, and budget ranges; a weather table including fields for time, location, temperature, humidity, precipitation probability, and a discretized weather category; a recipe table including fields for cuisine type, dish category, caloric estimation, ingredient list, cooking time, and difficulty level; and a product table including fields for product category, ingredient mapping key, unit price, store identifier, stock level, and online link.

The server integrates these heterogeneous data sources into a unified feature representation that is suitable for consumption by a generative AI model. The server constructs a feature vector and a text-based context for each user-specific request. The feature vector includes numerical and categorical values, such as encoded preference categories, normalized budget limits, health-condition embeddings, weather encodings, and recipe metadata scores. The text-based context includes descriptive sentences summarizing the user's state, environmental conditions, and candidate options. By combining a structured feature representation and a textual representation, the server enables efficient and accurate conditioning of the generative AI model.

The server employs a generative AI model implemented as a neural network, such as a transformer-based language model with multiple attention layers, feedforward layers, and layer-normalization units. The generative AI model is stored in a model repository and loaded into GPU memory or main memory for inference. The server configures the model with parameters such as the number of layers, attention heads, embedding dimension, and vocabulary size. The server trains or fine-tunes the generative AI model using a training dataset composed of synthetic and real dialog examples, meal plan descriptions, recipe-ingredient mappings, and explanation texts.

The server performs model training or fine-tuning offline by executing an optimization algorithm such as stochastic gradient descent or Adam to minimize a loss function such as a cross-entropy loss that compares model-generated tokens with ground-truth tokens. The server updates model weights according to gradients computed via backpropagation across the transformer layers. The server optionally performs data augmentation, such as paraphrasing prompt sentences, varying constraint combinations, and simulating different weather conditions, to improve the robustness of the generative AI model. As a result, the model acquires an internal representation that captures dependencies between personal data, context data, and meal-planning decisions.

During runtime, the server constructs a prompt sentence for the generative AI model in a deterministic and rule-based manner using the integrated feature representation. The server uses a prompt-construction module that follows a template and a set of non-conventional rules to place constraints and context in specific segments of the prompt sentence. For example, the server includes user constraints in a separate paragraph, weather details in another paragraph, and candidate recipes and ingredients in a well-structured list. This modular prompt structure causes the generative AI model to focus attention on constraint portions and candidate portions in a repeatable way, thereby improving inference stability and reducing the number of tokens needed to obtain a useful output.

The server generates, as an example of a prompt sentence:
“You are a meal planning assistant.

User profile: The user prefers Japanese and Chinese cuisines, is allergic to shrimp and peanuts, and requires a low-sodium dinner between 600 and 700 kcal.

Context: Today's weather at the user's location is hot (31 degrees Celsius) and humid with clear skies.

Candidate options: The following recipes are available: (1) chilled Chinese noodles, (2) cold tofu salad, (3) grilled chicken salad. Each recipe has associated ingredients and availability at nearby stores.

Task: Select one main dish and one side dish that best fit the user's health condition, preferences, and current weather. Explain the reasons, and list the required ingredients mapped to available products. Output the result in a clear, structured format.”

The server may generate other prompt sentences that emphasize different constraint conditions, such as available time, cooking skill level, and seasonal ingredients, while maintaining a consistent structure. For example, the server generates: “Based on the user's profile and today's hot weather, and using the provided recipes and product data, generate a dinner plan for tonight.

The user prefers light, cold dishes, has a nut allergy, and can spend at most 30 minutes cooking.

Weather is hot and humid.

Please output: dish names, reasons considering health and weather, and a detailed ingredient list with store information.”

By forming the prompt sentence according to such predetermined templates and non-standard ordering rules, the server reduces ambiguity and improves the efficiency of the generative AI model. This differs from human-like free-form requests because the prompt sentence is optimized for machine parsing and attention allocation rather than human readability alone.

The server receives an output from the generative AI model in the form of natural language text that adheres to the requested structure. The server parses the output using a parsing module that detects labeled sections, such as “Main dish,” “Side dish,” “Reason,” and “Ingredients.” The server uses a combination of pattern matching and token-level checks to extract ingredients, quantities, and optional nutrition-related information. If the prompt sentence requires a structured output format, the server enforces strict section headers and delimiter patterns, thereby enabling deterministic parsing and reducing post-processing complexity.

The server associates ingredient information from the model output with product information data stored in the database. The server uses mapping rules that compare normalized ingredient names with ingredient keys in the product table, optionally applying fuzzy matching algorithms and synonym dictionaries. The server selects product records that satisfy additional constraints, such as stock availability and price range. The server then generates purchase support information, which includes, for each ingredient, one or more eligible products with their store identifiers, estimated prices, and links for online ordering.

The server transmits the meal plan and the purchase support information to the terminal over the network. The terminal receives this structured output and renders a user interface that displays dish names, reasons for recommendation, ingredient lists, and available purchase options. The terminal uses layout components and local rendering libraries to present the information. The terminal may, for example, show a message such as:
“Today is hot, so we recommend chilled Chinese noodles and cold tofu salad. These dishes fit your low-sodium requirement and avoid shrimp and peanuts. Required ingredients and nearby purchase options are listed below.”

The user views the recommended meal plan on the terminal and optionally interacts with the purchase support interface by selecting items, marking purchased ingredients, or following online links. The terminal acquires user evaluation information, such as explicit ratings, likes or dislikes, and text comments, as well as behavior history information, such as which recommendations were opened, which purchase links were followed, and which dishes were repeatedly chosen or ignored.

The server collects this evaluation information and behavior history information and updates internal data structures accordingly. For example, the server adjusts preference vectors in the personal data by increasing weights for frequently selected cuisines and decreasing weights for seldom chosen dishes. The server also updates parameters that control prompt generation, such as relative emphasis on cost versus health metrics, or preference for cold versus hot dishes under specific weather categories. These updates are recorded in a user-specific model, which is applied the next time the server constructs a prompt sentence.

The server, by using such feedback-driven updates, improves the efficiency of subsequent processing. Since the server captures stable preference patterns and effective constraints in a machine-readable form, the server can construct shorter and more focused prompt sentences that reduce the number of tokens processed by the generative AI model and decrease the frequency of re-invoking the model to correct unsatisfactory outputs. This improves processing speed and reduces computational resource usage.

The server further optimizes data management by caching frequently used user features, weather categories, and candidate recipes in an in-memory store. As a result, the server avoids repeated database queries and redundant data transformations for similar contextual situations. The use of consistent internal feature representations and fixed prompt templates reduces variation in model calls and simplifies error handling, thereby increasing system reliability.

From a technical perspective, the system does not merely automate a human meal-planning task. Instead, the system introduces specific computer-oriented mechanisms that improve the functioning of the computing environment itself. The use of unified feature vectors, structured prompt templates, and feedback-driven parameter adjustment improves model inference efficiency, reduces the size and redundancy of network payloads, and decreases database access frequency. The server constrains generative processing to a well-defined search space derived from candidate recipes and product mappings, which reduces erroneous outputs and improves the accuracy of mapping ingredients to purchasable items.

In an alternative embodiment, the server uses multiple generative AI models or a combination of a generative AI model and a discriminative model. For instance, the server uses a first generative AI model to propose meal plans based on high-level constraints, and then uses a second, smaller model or a rule-based scoring function to verify nutritional constraints and budget adherence. The server may also replace or supplement the transformer-based model with a sequence-to-sequence model with attention, or a hybrid architecture combining recurrent units and attention modules. Training procedures are adapted correspondingly, with loss functions and regularization terms tailored to each architecture.

In another embodiment, the server modifies the prompt-construction algorithm to include an explicit ranking section, where the model is instructed to score candidate recipes on criteria such as cost, preparation time, and health alignment. The server then uses these scores to select or display meal plans in a particular order, thereby improving the interpretability and control of the recommendation process.

In a further embodiment, the server increases robustness against fluctuating external data, such as rapidly changing stock information or weather conditions, by assigning validity periods to cached feature sets and by selectively refreshing only those portions of the context that impact the prompt sentence. For instance, the server updates weather and stock features at a higher frequency than user preference features. This layered update strategy reduces unnecessary recomputation and network calls, producing a measurable reduction in average response time.

The terminal can be varied in form. In some embodiments, the terminal is a wearable device with a small display, in which case the server generates shorter, more concise explanation text. In other embodiments, the terminal is a large-screen device, and the server adds more detailed cooking instructions and nutritional breakdowns into the prompts and outputs. The same underlying generative AI model and prompt-construction principles are used, while presentation formatting is adjusted by the terminal.

By integrating these components and processes, the server, the terminal, and the user cooperate in a system that transforms raw contextual data into actionable, machine-optimized instructions for a generative AI model, and then into concrete purchase-support information and user interfaces. The system, through its specific data structures, neural network configuration, and prompt-engineering techniques, improves the technical performance of the computing environment, including faster response times, more accurate and constraint-compliant outputs, and reduced computational and communication overhead.

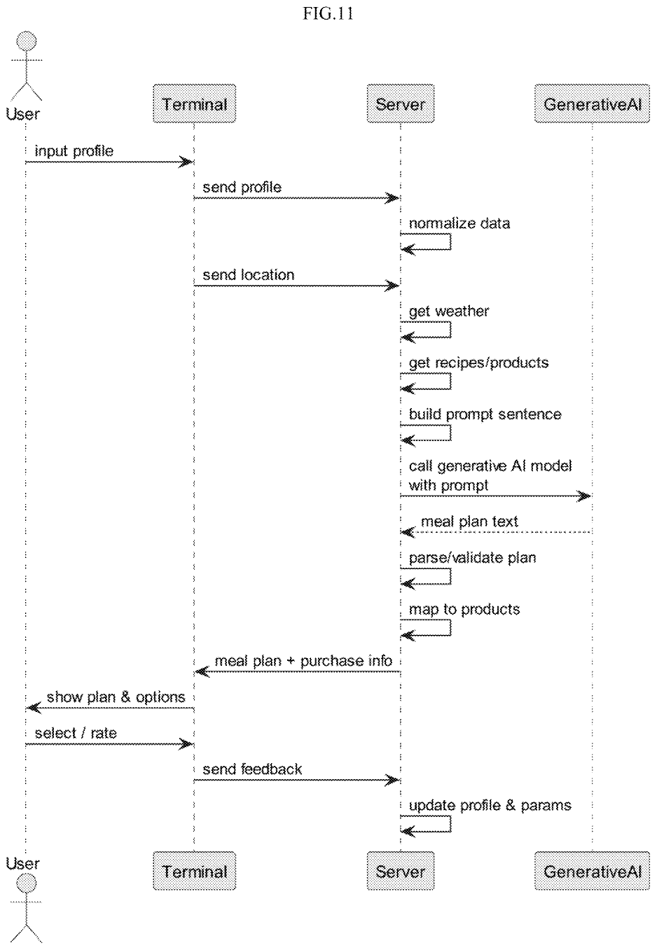

The following describes the processing flow using FIG. 11.

### Step 1:

The terminal acquires user profile information and transmits the information to the server.

The terminal displays input screens that allow the user to enter or select food preferences, allergy information, health conditions, budget range, usual meal time, cooking skill level, and available cooking time. The user operates the terminal via a touch panel or equivalent input device to select options and type text.

Input: Raw user input values (text, selections, toggles) on the terminal.

The terminal converts the input values into a structured data object, such as a set of key-value pairs, and performs basic validation (for example, checking that numeric fields contain numbers and that mandatory items are filled).

Output: A structured user profile dataset.

The terminal sends the structured user profile dataset to the server via a network using an HTTP request.

### Step 2:

The server receives and normalizes the personal data.

Input: The structured user profile dataset transmitted from the terminal.

The server parses the received dataset and stores the data in a user profile table in a database. The server then performs data normalization, including converting textual categories (for example, “low sodium”) into internal flags, transforming cuisine preferences into a preference vector, and encoding allergies as binary indicators. The server may also map free-text inputs to standard labels using a controlled vocabulary.

Output: A normalized personal data record and a corresponding internal feature representation.

### Step 3:

The server acquires weather data via an information acquisition unit and generates weather features.

Input: User location information (for example, latitude and longitude or region code) retrieved from the user profile or received from the terminal.

The server sends a request to an external weather information service through the information acquisition unit using a network protocol. The server receives weather data including current temperature, humidity, precipitation, and weather condition codes. The server parses the received weather data and converts the values into normalized numerical features, and also classifies the current weather into categories such as “hot,” “cold,” “humid,” or “rainy.”

Output: A weather data record and a weather feature set associated with the user and the current time.

### Step 4:

The server retrieves cooking information data and filters candidate recipes.

Input: The normalized personal data record and the weather feature set.

The server queries a recipe database using search conditions derived from the user's preferences, allergies, calorie range, cooking time, and the current weather category. The server receives multiple recipe records, each including a recipe identifier, dish name, ingredient list, estimated calories, cooking procedures, preparation time, and difficulty level. The server filters out recipes that contain allergen ingredients or that exceed the user's budget or time constraints. The server assigns scores to each remaining recipe based on alignment with user preferences and weather suitability.

Output: A set of candidate recipes with metadata and preliminary scores.

### Step 5:

The server retrieves product information data and maps ingredients to purchasable products.

Input: The set of candidate recipes and a product information database.

The server iterates over the ingredient list of each candidate recipe and performs lookups in the product information database using ingredient names and category keys. The server optionally applies text normalization and synonym matching to align ingredient names with product records. The server filters products based on stock status and price constraints, and, when multiple products are available for one ingredient, selects one or more optimal products according to predefined rules (for example, lowest price or nearest store).

Output: An ingredient-to-product mapping set for each candidate recipe, including product identifiers, store identifiers, prices, and online purchase links.

### Step 6:

The server constructs an integrated feature context and generates a prompt sentence for the generative AI model.

Input: The normalized personal data, the weather feature set, the candidate recipes, and the ingredient-to-product mapping set.

The server creates an internal representation that combines numerical features (for example, preference vectors and weather encodings) and textual summaries (for example, descriptions of the user's constraints and context). The server then uses a prompt-construction module to form a prompt sentence in a fixed template. The server inserts user constraints, weather description, a list of candidate recipes, and a summary of product availability into distinct sections of the prompt sentence.

Output: A complete prompt sentence and associated contextual data to be supplied to the generative AI model.

### Step 7:

The server invokes the generative AI model and obtains a meal plan.

Input: The prompt sentence and associated contextual data.

The server passes the prompt sentence and, optionally, compressed contextual information to the generative AI model hosted either locally or on a remote model-serving platform. The generative AI model, implemented as a neural network, processes the prompt sentence token by token and generates an output text that follows the requested structure, such as a main dish and side dish recommendation, reasons for the selection, and a structured ingredient list. The server receives the generated output from the model and stores it temporarily in memory for further processing.

Output: A model-generated meal plan text containing dish recommendations, explanations, and ingredient descriptions.

### Step 8:

The server parses the meal plan output and validates constraints.

Input: The model-generated meal plan text.

The server applies text parsing rules and patterns to detect labeled sections (for example, “Main dish,” “Side dish,” “Reasons,” “Ingredients”). The server extracts dish names, explanation sentences, and each ingredient entry from the text. The server checks whether any ingredient conflicts with the allergy information or violates nutritional or budget constraints. If a violation is detected, the server either removes the problematic recipe from consideration or modifies the candidate set and, when necessary, regenerates a prompt sentence to re-invoke the generative AI model.

Output: A validated meal plan object with dish entries and cleaned ingredient lists.

### Step 9:

The server associates the validated ingredient lists with product information and generates purchase support information.

Input: The validated meal plan object and the ingredient-to-product mapping set.

The server aligns each ingredient in the validated ingredient lists with corresponding product entries in the mapping set. The server calculates aggregated cost per dish and may compute additional indicators such as total estimated cost and potential savings. The server then builds purchase support information that includes, for each ingredient, product identifiers, store names, store locations, prices, and online purchase links.

Output: A combined meal plan and purchase support dataset ready for delivery to the terminal.

### Step 10:

The server transmits the meal plan and purchase support information to the terminal.

Input: The combined meal plan and purchase support dataset.

The server formats the dataset as a response payload and sends it to the terminal using a communication protocol. The payload includes all necessary fields for rendering dish names, explanatory text, ingredient details, and purchase options. The server may compress or otherwise optimize the payload to reduce network traffic.

Output: A structured response transmitted over the network to the terminal.

### Step 11:

The terminal receives and displays the meal plan and purchase support information.

Input: The structured response from the server.

The terminal parses the received data and constructs display elements such as lists, cards, and buttons. The terminal shows the user recommended dishes with explanations that reference the user's health condition and current weather. The terminal also displays, for each ingredient, the associated products, their prices, and purchase options, including links that can open external shopping applications or web pages.

Output: A user interface presenting the meal plan and purchase support information on the terminal's display.

### Step 12:

The user reviews the recommendations and interacts with the purchase options.

Input: The displayed meal plan and purchase support interface on the terminal.

The user reads the explanations, selects dishes to adopt for the meal, checks ingredient lists, and may tap on store options or online links to purchase items. The user may also mark some ingredients as already available at home or choose alternative store options presented on the terminal.

Output: User interaction events, such as selections, link activations, and ingredient status updates.

### Step 13:

The terminal collects evaluation information and behavior history and transmits these data to the server.

Input: The user interaction events generated during review and purchase actions.

The terminal converts user interactions into evaluation information (for example, a positive rating for a dish) and behavior history information (for example, which dishes were opened, which links were followed, and which were ignored). The terminal consolidates this information into structured records and sends these records to the server using a network request.

Output: Evaluation and behavior history records transmitted to the server.

### Step 14:

The server updates personal data and prompt-generation conditions based on the feedback.

Input: The evaluation and behavior history records from the terminal, and the existing personal data and configuration parameters.

The server analyzes the feedback by computing statistics such as frequency of selection of specific cuisines, correlation between weather conditions and accepted recommendations, and purchase completion rates. The server adjusts weighting factors in the personal preference vectors and updates prompt-generation parameters such as emphasis on cost, emphasis on preparation time, or preference for certain dish types under specific weather categories. The server writes the updated values back into the user profile database and any configuration store used for prompt construction.

Output: Updated personal data records and updated prompt-generation condition parameters.

### Step 15:

The server prepares for subsequent meal plan generation using the updated data.

Input: The updated personal data and updated prompt-generation condition parameters.

The server refreshes cached feature sets and replaces obsolete data structures with the updated representations. The server records the new parameter values to be used in future executions of the feature-integration and prompt-construction modules. As a result, the next time the user requests a meal plan, the server starts from a refined and more accurate internal state.

Output: A refined system state that enables more efficient and personalized execution of subsequent processing using the generative AI model and newly generated prompt sentences.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computerized meal recommendation and grocery ordering systems typically treat user attributes, environmental conditions, recipe content, and product availability as separate data silos. In many existing systems, a server independently recommends recipes based on static user profiles, or separately retrieves weather information and product information, without coherently integrating these heterogeneous data sources into a unified, machine-interpretable context for a generative AI model. As a result, such systems often fail to produce meal proposals that are contextually optimized in real time for user preferences, health conditions, environmental conditions, and actual product availability, thereby limiting the technical effectiveness of automated meal planning and ordering workflows.

Moreover, traditional systems frequently rely on manually crafted rule sets or fixed recommendation logic executed by the server. This rigid logic makes it difficult to dynamically adapt to complex combinations of factors such as changing weather, variable inventory conditions, user emotional states, evolving dietary goals, and temporal constraints (for example, preparation time during busy schedules). Consequently, the server-side computation is inefficient in generating personalized and operationally feasible meal plans, and the overall system may require substantial manual intervention by the user to adjust recommendations, search for ingredients, and complete purchase procedures.

Further, existing architectures generally do not exploit prompt-based interactions with generative AI models as a primary mechanism for orchestrating end-to-end processing, from acquisition of personal information and environmental information to generation of meal candidates, mapping to purchasable products, and initiation of delivery processes. In particular, many systems lack mechanisms for the processor to algorithmically construct structured prompt sentences that encode multiple technical constraints (including user budget, nutritional requirements, cooking skill, available preparation time, and delivery conditions), and to use the generative AI model as a flexible computational component within a tightly integrated transaction flow.

Additionally, there is insufficient support in conventional systems for inferring user emotional states or preference tendencies from historical interaction data and incorporating such inferred states into the prompt generation logic. Without this feedback loop, the server cannot effectively adjust subsequent meal generation processes in response to user reactions or long-term behavioral patterns, which reduces the precision and utility of the automated recommendation engine and increases the need for repetitive user input.

Accordingly, there is a need for an improved computer-implemented system that allows a processor to: (i) acquire and integrate personal information, environmental information, cooking information, and product information; (ii) algorithmically construct and refine prompt sentences for a generative AI model based on these integrated data; (iii) use the generative AI model as a computational engine to generate meal candidates, ingredient information, and responses to food-related questions; (iv) automatically search product information, generate product candidates, and coordinate purchase and delivery processes; and (v) continuously infer and incorporate user emotional states or preference tendencies so as to improve the technical performance, adaptability, and automation level of meal planning and grocery ordering workflows on the server side.

The specific processing by the specific processing unit 290 of the data processing device 12 in Application Example 1 is realized by the following means.

The present invention provides a server comprising a processor configured to acquire, from one or more storage units and via one or more communication networks, personal information including at least user preferences, allergy information, health condition information, budget information, cooking skill information, lifestyle schedule information, and temporal constraint information, environmental information including at least weather information and seasonality information, cooking information including at least cooking procedure information and ingredient information, and product information including at least product item information, price information, inventory information, and delivery condition information; to generate, on the basis of at least a portion of the personal information and the environmental information, a first prompt sentence to be input to a generative AI model, input the first prompt sentence to the generative AI model, and cause the generative AI model to generate one or more meal candidates and ingredient information corresponding to the one or more meal candidates; to transmit, to a terminal device, presentation information including the one or more meal candidates and the ingredient information, and to receive, from the terminal device, selection information indicating at least one selected meal candidate among the one or more meal candidates; to perform, on the basis of the selection information and the ingredient information, a search of the product information so as to acquire, for each ingredient included in the ingredient information, a plurality of product candidates and delivery information that satisfy at least one constraint condition represented by the personal information or the environmental information, and to transmit, to the terminal device, information representing the plurality of product candidates and the delivery information; to receive, from the terminal device, order confirmation information indicating at least a subset of the plurality of product candidates, and, on the basis of the order confirmation information, request one or more external services to execute a purchase process and a delivery process for the subset of the plurality of product candidates; and to estimate, on the basis of at least the personal information and a presentation history or selection history associated with the one or more meal candidates, at least one of a user emotional state and a user preference tendency, and to generate, on the basis of an estimation result, a second prompt sentence to be input to the generative AI model, to input the second prompt sentence to the generative AI model, and to cause the generative AI model to generate an additional meal candidate adapted to at least one of the user emotional state and the user preference tendency. This enables the server to technically improve the automation, personalization, and efficiency of meal planning and grocery ordering operations by using prompt-based interactions with a generative AI model that are dynamically constructed from integrated user context, environmental conditions, and product availability, while providing a feedback loop that refines subsequent processing based on inferred user emotional and preference states.

The term “processor” refers to a hardware-based or virtual information processing element, such as a central processing unit or a computing core, that executes instructions to perform logical operations, data processing, and control of other components in the system.

The term “storage unit” refers to any hardware or virtual component capable of storing data, including but not limited to a memory device, a database system, or a non-volatile storage device, that is accessible by the processor.

The term “personal information” refers to information related to a user that is used by the system to generate personalized outputs, including but not limited to user preferences, allergy information, health condition information, budget information, cooking skill information, lifestyle schedule information, and temporal constraint information.

The term “user preferences” refers to information indicating a user's likes, dislikes, or priorities regarding meals, food types, cuisines, flavors, or other dining-related choices.

The term “allergy information” refers to information indicating substances, ingredients, or food categories that a user must avoid due to allergic reactions or intolerances.

The term “health condition information” refers to information indicating a user's physical or medical status, including but not limited to dietary restrictions, ongoing health issues, or wellness goals that affect meal selection.

The term “budget information” refers to information indicating monetary limits or cost-related constraints for purchasing food items or ingredients for a user.

The term “cooking skill information” refers to information indicating a user's level of competence or experience in performing cooking tasks, which may influence the complexity or type of proposed meal candidates.

The term “lifestyle schedule information” refers to information indicating patterns of a user's daily activities, availability, or time allocation, including work schedules, commuting times, and typical meal times.

The term “temporal constraint information” refers to information indicating time-related limitations or requirements, including but not limited to maximum allowable meal preparation time or desired eating time.

The term “environmental information” refers to contextual information related to the user's environment, including but not limited to weather information, seasonality information, and other external conditions that may influence meal selection.

The term “weather information” refers to information indicating meteorological conditions, including but not limited to temperature, humidity, precipitation, and sky conditions, for a location associated with the user.

The term “seasonality information” refers to information indicating a time period within a year, such as a season or month, and optionally associated seasonal factors such as typical climate, customary foods, or seasonal ingredients.

The term “cooking information” refers to information describing how to prepare a meal, including at least cooking procedure information and ingredient information.

The term “cooking procedure information” refers to information indicating a sequence of operations or steps for preparing a meal, including but not limited to preparation steps, cooking times, and cooking methods.

The term “ingredient information” refers to information indicating materials used for meal preparation, including types of food items, quantities, and any associated properties such as freshness or form.

The term “product information” refers to information related to purchasable goods that correspond to ingredients or food items, including but not limited to product item information, price information, inventory information, and delivery condition information.

The term “product item information” refers to information describing an individual commercial product, including but not limited to a product identifier, a product name, a description, a unit size, and a category.

The term “price information” refers to information indicating a monetary cost associated with a product or service, including but not limited to unit price, discounts, and total estimated cost.

The term “inventory information” refers to information indicating stock availability of a product, including but not limited to quantity on hand, in-stock or out-of-stock status, and replenishment status.

The term “delivery condition information” refers to information indicating parameters or constraints for delivering products to a user, including but not limited to delivery time windows, shipping methods, serviceable areas, fees, and estimated delivery times.

The term “generative AI model” refers to an information processing model based on machine learning that generates new output data, such as text or structured information, in response to input data, and that produces meal candidates, ingredient information, or answers to questions by probabilistic or learned inference.

The term “prompt sentence” refers to an input expression, including text or structured text, that is constructed by the processor and provided to the generative AI model to specify a context, a constraint, or a task to be performed by the generative AI model.

The term “meal candidate” refers to information representing a proposed meal, including at least a dish name, a description, and associated ingredient information, that is generated or selected by the system for potential presentation to the user.

The term “presentation information” refers to information formatted for display or output to a terminal device, including but not limited to one or more meal candidates, ingredient information, product candidates, and related contextual details.

The term “terminal device” refers to an end-user computing device, including but not limited to a mobile communication apparatus, a portable information processing apparatus, or a fixed information processing apparatus, that communicates with the server and presents information to the user.

The term “selection information” refers to information received from the terminal device indicating a user's selection of at least one meal candidate or product candidate among those presented.

The term “product candidate” refers to a specific product entry selected or identified from product information as a potential match for an ingredient or group of ingredients required for a meal candidate.

The term “order confirmation information” refers to information received from the terminal device indicating that the user has confirmed purchasing at least one product candidate, including selected items, quantities, and optionally delivery and payment details.

The term “external service” refers to a system or service distinct from the server that provides a function such as product ordering, payment processing, or delivery management, and that is accessed by the server via a communication network.

The term “purchase process” refers to a sequence of operations performed by the server and/or an external service to complete a transaction for one or more product candidates, including but not limited to order placement, payment authorization, and order registration.

The term “delivery process” refers to a sequence of operations performed by the server and/or an external service to arrange and execute shipment of purchased products to a user-designated location.

The term “user emotional state” refers to an inferred or estimated state of a user's emotions, such as satisfaction, dissatisfaction, stress, relaxation, or other mood-related conditions, that may influence meal preferences or interaction behavior.

The term “user preference tendency” refers to an inferred or estimated pattern in a user's choices or behavior over time, such as a tendency to favor certain cuisines, nutritional profiles, or preparation times, derived from past interactions.

The term “presentation history” refers to information indicating which meal candidates or related content have been previously presented to the user, including timing and context of each presentation.

The term “selection history” refers to information indicating which meal candidates, product candidates, or recommendations have been selected or accepted by the user in the past, including frequency and timing of such selections.

The term “question information” refers to information representing an inquiry provided by a user via the terminal device, including but not limited to questions related to food, nutrition, cooking methods, storage methods, hygiene management, or lifestyle habits.

The term “response information” refers to information generated by the generative AI model in response to question information, and provided by the server to the terminal device as an answer or explanation.

In one embodiment, a server cooperates with one or more terminal devices operated by a user to implement the claimed system. The server is implemented on a computing platform such as a cloud-based virtual machine or a physical server including at least one processor, a main memory, a non-volatile storage device, and a network interface. The server executes an operating system, a web application framework, a database management system, and a machine learning runtime environment. For example, the server can execute a Unix-like operating system, a web framework such as a Python-based framework, a relational database management system such as a SQL-based database, and a machine learning stack capable of performing inference with a generative AI model. The terminal is implemented as a mobile communication device, such as a smartphone or tablet, or as a personal computer, executing an application framework such as a cross-platform mobile framework or a web browser.

The server stores, in one or more storage units, structured records representing personal information, environmental information, cooking information, and product information. The server represents the personal information in a normalized relational form, for example with tables or records including fields for user identifier, preferences, allergy information, health condition information, budget information, cooking skill information, lifestyle schedule information, and temporal constraint information. The server represents the environmental information in records including fields for geographic location, date and time, temperature, humidity, weather condition codes, and seasonality attributes. The server represents the cooking information in records including fields for recipe identifier, dish name, ingredient identifiers, ingredient quantities, cooking time, and multi-step cooking procedure descriptions. The server represents the product information in records including fields for product identifier, product name, unit size, price, currency, inventory level, supplier identifier, and delivery condition attributes such as service area and available time windows.

The server uses a generative AI model as a neural-network-based text generation engine. In one embodiment, the generative AI model is implemented as a transformer-based neural network comprising a stack of self-attention layers and feed-forward layers with learned weight matrices, layer normalization, and residual connections. The server maintains, within its machine learning runtime, a parameter set including token embedding matrices, positional embedding matrices, attention weight matrices, and output projection matrices. The server stores model hyperparameters such as number of layers, number of attention heads, hidden dimension size, activation functions, and maximum context length. The server executes inference with this generative AI model by encoding an input prompt sentence into a sequence of tokens, performing multi-head attention and feed-forward transformations layer by layer, and computing probability distributions over output tokens using a softmax operation. The server selects output tokens according to probabilities modified by temperature, top-k, or top-p sampling, thereby generating an output sequence representing a meal candidate description, ingredient list, or question response.

The server generates a prompt sentence as a structured natural language input to the generative AI model. The server constructs this prompt sentence by combining multiple categories of data into a single textual context using deterministic concatenation rules. For example, the server can generate a prompt sentence of the following form:
User preferences: Japanese food
Allergies: dairy products
Health condition: dieting
Budget: moderate
Cooking skill: beginner
Lifestyle schedule: busy weekday evening
Meal preparation time: within 30 minutes
Weather: 30° C., sunny
Please act as a professional nutritionist and chef.
Please generate a healthy, low-calorie dinner menu suitable for hot weather and a busy weekday evening.
Output the menu name, a brief description, and a structured list of ingredients with approximate quantities.

The server uses this prompt sentence as an input sequence to the generative AI model. The server tokenizes the prompt sentence using a tokenizer consistent with the model's vocabulary, maps tokens to embedding vectors, and processes them through the transformer layers. The server thus performs a concrete sequence of tensor multiplications, attention score calculations, and non-linear activations to compute the output token sequence. This processing is not merely a conceptual “AI decision” but an explicit sequence of numerical operations executed on the processor and memory subsystem.

The server improves computer technology by encoding heterogeneous constraints into the prompt sentence using a data-driven rule set rather than a static rule engine. The server maps structured attributes (for example, budget thresholds, preparation time limits, or delivery windows) into explicit textual constraints in the prompt sentence. As a result, the generative AI model can internally learn multi-dimensional relationships between constraints and outputs, which allows the server to reduce the amount of iterative user interaction and significantly reduce the number of network round trips required to converge on an acceptable meal plan. This reduction in network traffic and repeated queries leads to a technical improvement in communication efficiency and system latency.

The server maintains a modular architecture for data processing. A profile module acquires and updates personal information from the terminal. A context module acquires environmental information from external services through the network interface and normalizes it into a standard data structure. A recipe module maintains cooking information and can filter recipes by dietary constraints. A product module acquires product information from external commerce services and maintains an index keyed by ingredient names and attributes. A prompt generation module converts structured data produced by these modules into a prompt sentence. A generative inference module executes generative AI model inference using hardware acceleration if available. A mapping module parses generated text into structured meal candidates and ingredient lists using pattern-based parsing and language model-assisted segmentation. A selection and ordering module aligns the ingredient lists with product candidates and orchestrates ordering with external services.

The server generates different categories of prompt sentences according to operational context. When the server generates meal candidates, the server constructs a prompt sentence including user preferences, allergies, health condition, budget, cooking skill, lifestyle schedule, meal preparation time, and environmental information. For instance, the server can generate a prompt sentence in the following form:
User preferences: Italian food
Allergies: nuts
Health condition: high blood pressure
Budget: low
Cooking skill: intermediate
Lifestyle schedule: working from home
Meal preparation time: within 45 minutes
Weather: 15° C., rainy
Please act as a professional nutritionist and chef.
Please propose a warm, low-salt Italian dinner menu that fits the above conditions.
Include the dish name, a short explanation, and a list of ingredients with approximate quantities.

When the server responds to question information related to food or health, the server constructs a prompt sentence including the question and optionally the user's health context. For example, the server can generate a prompt sentence as follows:
User health condition: high cholesterol
Question: What kind of dinner should I eat tonight to help manage my cholesterol while keeping preparation time under 30 minutes?
Please answer as a professional nutritionist.
Provide a brief explanation and a concrete example meal.

When the server adjusts recommendations based on user emotional state or preference tendency, the server first computes an estimated state by analyzing presentation history and selection history. The server maintains, for each user, a time-series record including a timestamp, identifiers of presented meal candidates, indicators of whether the user selected or skipped each candidate, and optionally user feedback levels. The server computes features such as frequency of acceptance for each cuisine type, average preparation time of accepted meals, and correlation between weather conditions and chosen dishes. The server may additionally apply a classifier model or a recurrent neural network to these features to estimate a user emotional state or preference tendency vector. The server then encodes the result into a prompt sentence, for example:
User preferences: Japanese food, light meals
Inferred emotional state: stressed
Inferred preference tendency: prefers quick, simple meals on weekdays
Weather: 28° C., cloudy
Please act as a professional nutritionist and chef.
Please generate a simple, light Japanese dinner menu that helps the user relax after a stressful day and can be prepared in less than 20 minutes.
Output the menu name, a short description focusing on relaxation, and a list of ingredients.

The server thus uses a non-conventional rule set in which estimated emotional state and preference tendency are explicitly inserted into the prompt sentence. This rule set is distinct from manual human decision-making and from fixed rule-based recommenders, and it modifies the model's attention to particular constraints in a systematic way, which leads to a measurable change in output distributions.

The terminal communicates with the server using an application-layer protocol over a network such as the Internet. The terminal displays presentation information including one or more meal candidates and ingredient lists generated by the server. The terminal accepts user input for selecting meal candidates, adjusting quantities, and confirming orders. The terminal sends selection information and order confirmation information to the server, but the terminal does not perform the core generative computation or data integration; these functions are concentrated at the server side for consistency and computational efficiency.

The user interacts with the terminal to input personal information, confirm or edit suggestions, and trigger ordering operations. The user may provide feedback such as explicit ratings or comments that the server stores and uses as additional features for estimating user emotional state and preference tendency. The user may also input free-form dietary questions, which the terminal forwards as question information to the server. The user thus indirectly influences the generative AI model behavior through both direct prompts and cumulative interaction history.

The server improves data management by representing user-related, environment-related, and product-related data in normalized structures and by linking them through foreign key relationships or equivalent indices. The server maintains an index mapping ingredient names and categories to sets of product identifiers. When the server receives ingredient information from the generative AI model, the server standardizes ingredient names through token normalization and string matching algorithms and then performs efficient lookups in the index to identify candidate products. This approach reduces computational complexity compared to naive full-text search and improves response time when mapping ingredients to purchasable products.

The server improves computation efficiency by reusing intermediate results and caching. The server can cache weather information for a region for a period shorter than a predetermined time threshold to avoid redundant external queries. The server can cache frequently used prompt templates and only update variable parts, such as numeric values for budget or preparation time, thereby reducing string construction overhead. The server can also cache generative AI outputs for similar contexts, enabling the server to respond quickly to repeated or similar requests while reducing inference load and network usage.

The server can be implemented in alternative embodiments. In one alternative, the server executes the generative AI model locally on dedicated hardware such as a graphics processing unit or a specialized accelerator. In another alternative, the server accesses a remote inference service through a secure network API while managing all data pre-processing and post-processing locally. In yet another alternative, the server combines a base generative AI model with an adapter layer or fine-tuned sub-model specializing in dietary planning, where the adapter modifies internal activations or attention patterns based on domain-specific training. In any of these embodiments, the server explicitly defines the generative AI model as a parameterized computational graph with a defined training method, such as gradient-based optimization using a cross-entropy loss function between predicted and actual tokens, and weight updates performed via backpropagation on pre-collected training corpora of recipes, nutritional descriptions, and user review texts.

The server enhances accuracy and reduces error by integrating safety and constraint checking after generative output. The server parses generated ingredient information and checks against allergy information, dietary restrictions derived from health condition information, and inventory constraints. If the generative output violates a hard constraint (for example, inclusion of an allergen or an ingredient that is not available in the user's region), the server automatically initiates an adjustment procedure. In one embodiment, the server generates a follow-up prompt sentence that explicitly states the constraint violation and requests the generative AI model to revise the menu while satisfying all constraints. This iterative constraint enforcement loop is implemented programmatically and operates faster and more consistently than manual revision by the user, thus providing a technical effect of reducing error rates and improving safety.

The server reduces communication load by embedding multiple operational objectives into a single prompt sentence rather than issuing multiple independent queries. For example, the server can encode both dietary constraints and emotional state information into a single prompt sentence, thereby obtaining an integrated meal candidate from a single inference pass. This reduces the number of interactions between the server and the generative AI model and also reduces the number of interactions between the server and the terminal, which in turn decreases overall network usage and improves response time.

The server can be extended to support different data sources and hardware configurations. In one embodiment, the server obtains environmental information not only from general-purpose weather services but also from local sensor devices, such as temperature or humidity sensors installed in a dwelling. In such a case, the server converts sensor outputs into environmental information records and uses them similarly in prompt sentence construction. In another embodiment, the server controls automated kitchen devices such as smart ovens or cooking appliances by emitting control signals based on generated cooking procedure information, thus connecting generative planning to actual device control in the physical environment.

By organizing operations around prompt sentence generation and generative AI model inference that are tightly integrated with structured data management and constraint checking, the server achieves improvements in processing speed, recommendation accuracy, and communication efficiency compared to conventional rule-based or simple retrieval-based systems. The integration of user emotional state inference and preference tendency modeling into the prompt generation process further refines outputs in a non-trivial computational manner, demonstrating that the system is not merely automating human decision-making but is improving the way a computer system represents, processes, and utilizes complex multi-dimensional context for real-world meal planning and food delivery tasks.

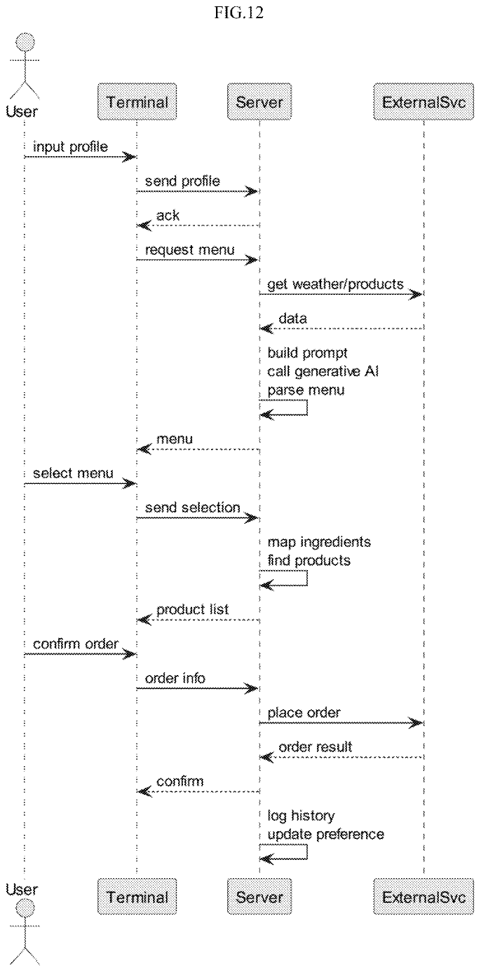

The following describes the processing flow using FIG. 12.

### Step 1:

The user operates the terminal and inputs personal information including at least user preferences, allergy information, health condition information, budget information, cooking skill information, lifestyle schedule information, and desired meal preparation time. The terminal receives this input via form fields, validates formats (for example, that preparation time is numeric and mandatory fields are not empty), and constructs a structured request. As input, the terminal uses raw user keystrokes and UI selections, and as output, the terminal generates a structured data object representing the personal information. The terminal then transmits this structured data object to the server over a network.

### Step 2:

The server receives the personal information from the terminal and stores it in a storage unit. As input, the server uses the structured personal information object, parses each field, and normalizes values (for example, mapping free-text cuisine names to standard category codes and converting budget ranges to numeric thresholds). The server performs data processing including validation, normalization, and mapping to relational database fields, and as output, the server writes one or more records to a database table keyed by a user identifier and confirms successful storage.

### Step 3:

The server acquires environmental information related to the user. As input, the server uses the user identifier and optionally a location parameter received from the terminal, and the server sends a request to one or more external information services (for example, a weather service) via the network. The server receives weather data including temperature, condition codes, and timestamps. The server performs data processing by parsing a response structure, converting condition codes into human-readable labels, calculating derived attributes such as “hot,” “cold,” or “rainy,” and mapping dates to seasonality categories. As output, the server generates an environmental information record and stores or caches it in the storage unit.

### Step 4:

The server acquires cooking information and product information relevant to possible meals. As input, the server uses the stored personal information and environmental information to filter an internal recipe database and to query external product information services. The server executes data operations such as SQL queries over a recipe table, applying constraints derived from allergies, health condition, and cooking skill level, and sends search queries to product information providers keyed by ingredient categories. As output, the server obtains a candidate set of recipes and a candidate set of product entries, each standardized into internal structures with identifiers, attribute fields, and relationships to ingredients.

### Step 5:

The server generates a first prompt sentence for a generative AI model. As input, the server uses the normalized personal information, the environmental information, and optionally filtered cooking information. The server executes a deterministic string construction procedure: it selects fixed template segments, inserts variable values (for example, temperature, health condition, budget, preparation time), and concatenates segments with explicit labels. This processing converts structured data fields into a coherent natural language context. As output, the server produces a prompt sentence such as:
User preferences: Japanese food
Allergies: dairy products
Health condition: dieting
Budget: moderate
Cooking skill: beginner
Lifestyle schedule: busy weekday evening
Meal preparation time: within 30 minutes
Weather: 30° C., sunny
Please act as a professional nutritionist and chef.
Please generate a healthy, low-calorie dinner menu suitable for hot weather and a busy weekday evening.
Output the menu name, a brief description, and a structured list of ingredients with approximate quantities.

### Step 6:

The server executes inference on the generative AI model using the prompt sentence. As input, the server uses the prompt sentence and passes it through a tokenizer and embedding module of a transformer-based neural network. The server performs data processing including tokenization (mapping characters to token identifiers), vector embedding lookup, multi-head attention computations, matrix multiplications, non-linear activations, and softmax probability calculations across multiple network layers. As output, the server obtains a sequence of tokens representing a generated text response, which the server reconstructs into a textual description of at least one meal candidate including a dish name, a description, and an ingredient list.

### Step 7:

The server parses the generated text and converts it into a structured meal candidate representation. As input, the server uses the generated text sequence from the generative AI model. The server performs string processing such as line splitting, pattern matching for headings (for example, “Menu name:” or “Ingredients:”), and extraction of ingredient entries and quantities. The server may also apply language model-assisted segmentation to disambiguate ingredient lines. As output, the server produces a data structure containing fields for menu name, description, and a list of ingredient items, each with a name and a quantity string, and stores this structure in the storage unit with an association to the user identifier.

### Step 8:

The server transmits presentation information to the terminal. As input, the server uses the structured meal candidate data structure and optionally mapped metadata (for example, estimated calorie range). The server performs formatting operations to organize the data into a response object appropriate for the terminal's user interface, such as grouping ingredients and attaching tags (for example, “low-calorie” or “quick-cooking”). As output, the server sends a response containing at least the meal candidate name, description, and ingredient list to the terminal via a network protocol.

### Step 9:

The terminal receives and displays the meal candidate to the user. As input, the terminal uses the presentation information from the server and parses the structured data. The terminal performs display operations, including rendering the meal name as a title, showing the description as a text block, and presenting each ingredient as an item in a list, optionally with icons or quantity labels. As output, the terminal generates a graphical user interface that allows the user to visually review the suggested meal and interact with controls such as “Select this menu” and “Order ingredients.”

### Step 10:

The user selects a meal candidate or initiates an ordering operation. As input, the user observes the displayed meal candidate on the terminal and operates one or more user interface elements, such as tapping on a “Select” button or an “Order” button. The user may also adjust ingredient quantities or deselect certain items through the terminal's interface. As output, the user causes the terminal to generate selection information or order initiation information that encodes which meal candidate is chosen and which ingredients should be ordered.

### Step 11:

The terminal sends selection information to the server. As input, the terminal uses the user's interaction result, including identifiers for the selected meal candidate and any adjusted ingredient quantities. The terminal performs data packaging, assembling a structured message that includes the user identifier, meal candidate identifier, and selected ingredient parameters. As output, the terminal transmits this selection information to the server via the network.

### Step 12:

The server maps the selected ingredients to product candidates. As input, the server uses the stored meal candidate structure and the received selection information, including ingredient names and desired quantities. The server performs data processing by normalizing ingredient names (for example, lowercasing, removing extra descriptors), mapping them to standardized ingredient categories, and querying the product information index. The server executes search algorithms that match ingredient categories and quantity ranges to product entries with suitable unit sizes, prices, and inventory levels. As output, the server generates, for each ingredient, a list of product candidates with associated product identifiers, price information, and delivery condition information.

### Step 13:

The server transmits product candidate information to the terminal. As input, the server uses the per-ingredient product candidate lists and relevant delivery information. The server formats this data into a response structure that groups products by ingredient and includes necessary attributes for user decision-making, such as price, brand type, and estimated delivery time. As output, the server sends this product candidate information to the terminal over the network.

### Step 14:

The terminal renders product candidates and accepts order confirmation. As input, the terminal uses the structured product candidate information received from the server. The terminal performs UI rendering operations, such as displaying multiple selectable product options for each ingredient and presenting total cost and delivery window summaries. The terminal then accepts user interactions where the user chooses specific products and confirms or cancels the order. As output, the terminal generates order confirmation information that specifies selected product identifiers, quantities, and any final delivery preferences.

### Step 15:

The server processes the order confirmation and initiates external purchase and delivery processes. As input, the server uses the order confirmation information from the terminal, including selected product identifiers and delivery preferences. The server performs data validation to confirm that products are still available and that the total cost and delivery conditions comply with the budget and constraints stored in personal information. The server then constructs one or more requests to external services for ordering and logistics, encoding product identifiers, quantities, delivery addresses, and payment tokens. As output, the server sends these requests to one or more external services and receives order identifiers, status codes, and estimated delivery times, which the server stores and later returns to the terminal.

### Step 16:

The server records interaction history and estimates user emotional state and preference tendency. As input, the server uses the complete trace of interactions, including which meal candidates were generated, which were presented, which were selected or rejected, and what ordering decisions were made. The server stores this data as presentation history and selection history in the storage unit. The server then computes features such as acceptance ratios per cuisine type, correlation between weather conditions and chosen meals, and time-of-day patterns. The server may apply a statistical model or a neural classifier to these features to derive an estimated user emotional state and a user preference tendency vector. As output, the server produces an updated user profile extension that includes these inferred attributes.

### Step 17:

The server generates a second prompt sentence that incorporates inferred emotional state and preference tendency. As input, the server uses the extended user profile including inferred emotional state, preference tendencies, and the current environmental information. The server executes a similar string construction process as in the first prompt generation but adds explicit mention of the inferred states. The server, for example, creates a prompt sentence such as:
User preferences: Japanese food, light meals
Inferred emotional state: stressed
Inferred preference tendency: prefers quick, simple meals on weekdays
Weather: 28° C., cloudy
Please act as a professional nutritionist and chef.
Please generate a simple, light Japanese dinner menu that helps the user relax after a stressful day and can be prepared in less than 20 minutes.
Output the menu name, a short description focusing on relaxation, and a list of ingredients.

The server thus converts internal inferred attributes into a human-readable constraint set embedded in the prompt sentence. As output, the server produces this second prompt sentence and feeds it as input to the generative AI model.

### Step 18:

The server performs generative AI inference again based on the second prompt sentence and updates recommendations. As input, the server uses the second prompt sentence and repeats the inference operations described previously, including tokenization, embedding, transformer-layer computation, and token sampling. The server generates a refined meal candidate that is adapted to the user's emotional state and observed preference patterns. The server parses the model's output into a structured form, stores it, and transmits updated presentation information to the terminal. As output, the server delivers an improved, context-sensitive meal proposal that is different from and typically more suitable than the initial proposal, thereby closing the feedback loop between user interaction, computational inference, and generative planning.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented meal recommendation systems generally rely on static rule engines or simple filtering of database records based on user-specified conditions such as budget, cooking time, or dietary preference. In such systems, the processor typically applies fixed decision trees or basic query conditions to structured data, and returns a limited set of candidate recipes. These approaches suffer from several technical deficiencies.

First, conventional systems do not efficiently integrate heterogeneous data types, namely structured database records and unstructured natural language text generated by generative AI models. As a result, the processor either ignores rich contextual information that could be provided by generative models or must process it through ad hoc, application-specific code, which increases processing latency and memory consumption and leads to inconsistent output formats. This limits the scalability and responsiveness of the system, particularly when operating on resource-constrained server environments handling large numbers of concurrent users.

Second, conventional systems lack a robust mechanism for dynamically generating prompt sentences to generative AI models that are tightly constrained by machine-readable user profiles and database-derived constraints. Prompt sentences are often manually designed or loosely coupled to the underlying structured data, which can cause the generative AI model to produce suggestions that violate core constraints such as user budget, nutritional limitations, or available preparation time. This leads to an increased need for manual post-processing or repeated API calls, thereby degrading computational efficiency and system throughput.

Third, many existing systems do not provide an integrated computational loop that automatically re-evaluates generative AI outputs against nutritional and cost models at the server level. Without such a loop, the server cannot automatically discard or down-rank proposals that fail to satisfy technical constraints derived from user profiles and nutritional computation modules. Consequently, the server is forced either to rely on user-side judgment or to apply simplistic keyword-based checks, which are computationally inefficient and error-prone when executed at scale.

Fourth, conventional architectures often treat interactive question answering regarding ingredients, substitutions, storage methods, and food safety as a separate subsystem or as a direct, unstructured chat between a user terminal and an external AI service. This separation prevents the server from systematically leveraging existing structured cooking-related information and personal profiles in the formation of prompt sentences, and from normalizing the AI responses into structured formats suitable for further computation and caching. This leads to redundant calls to external AI services, inconsistent answer quality, and increased network and computation overhead.

Therefore, there is a need for an improved computer-implemented system and processing method in which a processor, in cooperation with a storage device and a generative AI model, (i) programmatically generates structured prompt sentences from machine-readable personal information and cooking-related data, (ii) integrates and normalizes AI-generated cooking proposals with database-derived candidate data, (iii) performs server-side nutritional and constraint verification in a closed computational loop, and (iv) provides structured, display-ready output to a user terminal. Such a system should improve computational efficiency, response consistency, and scalability of meal recommendation processing, and should reduce the processing burden on user terminals and application-layer code.

The specific processing by the specific processing unit 290 of the data processing device 12 in Example 2 is realized by the following means.

The present invention provides a server comprising a processor and a storage device, the processor being configured to execute instructions that cause the processor to acquire personal information regarding a lifestyle of a user, including at least budget information, health condition information, nutritional condition information, seasonal condition information, skill level information, and meal preparation time information, via an input apparatus; query structured cooking-related information in the storage device based on the personal information and associated identification information; obtain, as structured cooking-related information, data including at least cost information, cooking time information, cooking procedure information, ingredient attribute information, and nutritional component information; perform numerical and logical operations according to a program stored in a recording medium to apply filtering and ranking to the cooking-related information in accordance with at least a cost condition, a time condition, a nutritional condition, and a user skill condition to generate candidate cooking information; generate a prompt sentence, as machine-readable text data for input to a generative AI model, based on the personal information and the candidate cooking information; transmit the prompt sentence to an internal or external generative AI model, receive response text from the generative AI model, parse the response text to extract cooking proposal information, integrate the cooking proposal information with the candidate cooking information, and convert integrated cooking proposal information into structured response data to be transmitted to a user terminal as display information. This enables the server to implement an end-to-end, machine-controlled data processing pipeline that unifies structured database querying with generative AI interaction, improves computational efficiency and consistency of meal recommendations by automatically constraining and normalizing AI outputs, and provides scalable, low-latency delivery of nutritionally and contextually appropriate cooking proposals to user terminals.

The term “personal information” refers to machine-readable data representing attributes of a user, including at least budget information, health condition information, nutritional condition information, seasonal condition information, skill level information, and meal preparation time information.

The term “budget information” refers to data indicating a constraint on an allowable expenditure for food or meals within a predetermined period or per meal.

The term “health condition information” refers to data indicating a physiological or medical state of a user, including at least presence or absence of specific diseases, allergies, dietary restrictions, or health goals.

The term “nutritional condition information” refers to data indicating desired or restricted intake levels of nutrients, such as energy, protein, fat, carbohydrates, salt, vitamins, and minerals.

The term “seasonal condition information” refers to data indicating availability or preference of ingredients or dishes according to a season, climate, or time period.

The term “skill level information” refers to data indicating a cooking proficiency level of a user, including at least a beginner level, an intermediate level, and an advanced level.

The term “meal preparation time information” refers to data indicating an allowable time period for preparing and cooking one or more dishes.

The term “identification information” refers to data used to uniquely or semantically associate personal information with a particular user profile, session, or record in a storage device.

The term “storage device” refers to a physical or logical data storage apparatus, including at least a non-volatile memory, a database system, or a distributed storage system, configured to store structured or unstructured data.

The term “structured information” refers to data organized according to a predetermined schema, such as tables, records, fields, or hierarchical data structures, enabling access by key, index, or query operations.

The term “cooking-related information” refers to structured information associated with food or cooking, including at least cost information, cooking time information, cooking procedure information, ingredient attribute information, and nutritional component information.

The term “cost information” refers to data indicating an estimated or actual monetary cost associated with ingredients or preparation of a recipe.

The term “cooking time information” refers to data indicating an estimated or actual time required to prepare and cook a recipe, including at least preparation time and heating time.

The term “cooking procedure information” refers to data indicating ordered steps, actions, or operations for preparing and cooking one or more dishes.

The term “ingredient attribute information” refers to data indicating properties of food ingredients, including at least type, quantity, unit, origin, freshness, and category.

The term “nutritional component information” refers to data indicating amounts or ratios of nutritional substances contained in ingredients or dishes, including at least energy, macronutrients, and micronutrients.

The term “data management apparatus” refers to a computing apparatus or system configured to manage, store, and serve structured information, such as a database server, a data warehouse, or a cloud-based data service.

The term “recording medium” refers to a non-transitory physical medium on which program instructions, data structures, or configuration data are stored, including at least semiconductor memory, magnetic storage, or optical storage.

The term “filtering processing” refers to a computational operation that removes or excludes data items that do not satisfy one or more predefined conditions, such as cost, time, nutritional constraints, or skill constraints.

The term “ranking processing” refers to a computational operation that assigns scores or priorities to data items based on one or more evaluation criteria and orders the data items according to the scores or priorities.

The term “candidate cooking information” refers to a subset of cooking-related information selected and ordered by filtering processing and ranking processing, and considered as potential recommendations for a user.

The term “generative AI model” refers to a machine learning model configured to generate text or other content in response to input data, the model being trained on large-scale datasets and implemented on one or more computing apparatuses.

The term “prompt sentence” refers to text data that encodes instructions, conditions, or contextual information supplied as input to a generative AI model to control or guide generation of output content.

The term “response text” refers to one or more text sequences generated by a generative AI model in response to a prompt sentence.

The term “cooking proposal information” refers to structured or semi-structured data derived from response text, including at least proposed recipes, ingredient lists, preparation steps, and explanatory comments.

The term “integrated cooking proposal information” refers to data obtained by combining cooking proposal information with candidate cooking information according to predetermined integration rules or data schemas.

The term “structured data format” refers to a machine-readable format that represents data with explicit structure, such as a JavaScript Object Notation (JSON) format, an Extensible Markup Language (XML) format, or a relational record format.

The term “response data” refers to structured data generated by a server for transmission to a user terminal, the data including at least integrated cooking proposal information and associated metadata.

The term “display information” refers to data encoded in a format suitable for presentation on an output interface of a user terminal, including at least text, numerical values, lists, and structured layouts of cooking-related information.

The term “user terminal” refers to an electronic apparatus operated by a user, including at least a mobile terminal, a desktop terminal, or a web-enabled device, configured to send input information to a server and present display information to the user.

The term “nutrition value calculation program” refers to software instructions that, when executed by a processor, compute or estimate nutritional indices, including at least energy and nutrient amounts, based on ingredient information and nutritional component information.

The term “intake energy” refers to a calculated or estimated amount of energy, typically expressed in kilocalories or kilojoules, associated with consumption of one or more dishes.

The term “nutritional indices” refers to one or more numerical indicators characterizing nutritional properties of food, including at least macronutrient distribution, micronutrient levels, and compliance with nutritional guidelines.

The term “final cooking recommendation information” refers to information representing one or more cooking proposals that remain after recalculation of nutritional indices and exclusion or down-ranking of proposals that do not satisfy conditions included in personal information.

The term “interactive prompt sentence” refers to a prompt sentence configured for interactive or conversational use, including at least an inquiry sentence regarding food or cooking, and adapted to elicit question-and-answer type responses from a generative AI model.

The term “answer information” refers to information generated by a generative AI model in response to an interactive prompt sentence, including at least substitute ingredient information, simplified cooking procedure information, storage method information, and safety-related information.

In one or more embodiments, the server, the terminal, and the user cooperate to implement a meal recommendation system that integrates structured data processing and interaction with a generative AI model. The system is configured so that a person having ordinary skill in the art can implement the invention on general-purpose computing hardware using known software platforms.

The server includes at least one processor, a main memory, a non-volatile storage device, a network interface, and one or more databases. The server executes an operating system, such as a generic server operating system, and application software implemented for example in a general-purpose programming language. The server communicates with the terminal via a network such as the Internet using a communication protocol such as HTTPS. The terminal includes at least one processor, a memory, a display device, an input device such as a touch panel or keyboard, and a communication interface. The terminal executes a client application or a web browser to enable the user to interact with the server.

The server stores a program in the storage device. The program, when executed by the processor of the server, causes the server to perform data acquisition, data transformation, numerical computation, and communication with a generative AI model according to the structures and flows described in the claims. The server uses a database management system, such as a relational database system, to store structured information representing personal information, cooking-related information, and logs of interactions with the generative AI model. The server uses schemas including tables for users, user profiles, recipes, ingredients, nutritional values, and AI-generated proposals. For example, the server stores a recipe record as a row having fields such as recipe identifier, title, list of ingredient identifiers, preparation time, estimated cost, and references to nutritional components. The server stores ingredient records having attributes including ingredient identifier, category, unit price, typical serving size, and links to nutritional composition records.

The terminal executes a client program that presents input forms to the user. The user uses the terminal to input personal information, such as a budget, a health condition, desired nutritional conditions, seasonal preferences, a cooking skill level, and an allowable meal preparation time. The terminal converts this personal information into structured key-value pairs and transmits them to the server via a secure network connection. The server receives the personal information and stores it in appropriate database tables as structured data. The server then uses this personal information as parameter values for subsequent data queries and computations.

The server uses a data access layer to construct and execute database queries against the storage device. The server uses indexed columns and join operations to efficiently retrieve cooking-related information that matches coarse constraints, such as budget ranges and cooking time ranges. The server loads retrieved records into in-memory data structures in the main memory. The server then applies a filtering algorithm and a ranking algorithm implemented as numerical and logical operations. In one embodiment, the server represents each candidate recipe as a feature vector including normalized values for estimated cost, cooking time, caloric content, protein amount, fat amount, carbohydrate amount, sodium amount, and a skill-level suitability score. The server calculates the feature vector by combining values from the recipe table, the ingredient table, and the nutritional composition table. The server uses a predetermined weighting scheme to compute a composite score. For instance, the server multiplies each feature by a weight that depends on the importance of the corresponding constraint for the user (e.g., stronger weight on sodium for users with hypertension). The server then sums the weighted features to obtain a scalar score for each candidate recipe.

The server stores or configures the weighting scheme in the storage device so that it can be adapted for different user segments or use cases. The server performs sorting operations on the candidate list in memory using efficient sorting algorithms, thus obtaining a ranked list of candidate cooking information. The server discards candidates that do not meet hard constraints, such as exceeding the maximum budget or maximum cooking time. The remaining candidates constitute candidate cooking information as defined in the claims.

The server generates a prompt sentence as text data for input to the generative AI model. The server uses a template engine or formatted string generation routine that inserts concrete values from the personal information and the candidate cooking information into parameterized text templates. The server, for example, generates a prompt sentence such as:
“User profile: daily budget 2,000 JPY, wants to reduce salt intake, prefers high-protein and low-fat meals. Cooking skill is beginner and available cooking time is 30 minutes per meal. Based on this profile and the following candidate recipes from our database: grilled chicken salad with seasonal vegetables, tofu and vegetable stir-fry, tomato and bean soup with whole grain bread, propose 3 concrete dinner recipes that fit within the budget, have low sodium, are high in protein, and can be prepared by a beginner within 30 minutes. For each recipe, provide ingredients, quantities, step-by-step instructions, estimated cooking time, and an explanation of nutritional benefits.”

In another example, the server generates a weekly-plan prompt sentence such as:
“Budget: 20,000 JPY per week. Health goal: healthy meals focused on vegetables and balanced nutrition. Skill level: beginner. Time constraint: all recipes must be cookable within 30 minutes. Please propose a 7-day dinner plan with one recipe per day that meets these conditions. For each day, output the recipe name, ingredients with quantities, estimated cost, preparation time, and a short tip for beginners.”

The server transmits such a prompt sentence to a generative AI model via a network interface. The generative AI model is implemented on an external or internal computing apparatus that includes a plurality of processing units such as graphics processing units or tensor processing units. In one embodiment, the generative AI model is a transformer-based neural network having multiple attention layers, feed-forward layers, and layer-normalization layers. The server or a model provider trains the generative AI model on large-scale corpora including cooking-related texts, ingredient descriptions, and instructions, using supervised learning and, optionally, reinforcement learning from human feedback. During training, the generative AI model receives input tokens representing prompt sentences encoded using a tokenizer, computes hidden representations using multi-head self-attention, and predicts output tokens. The model provider uses a loss function such as cross-entropy loss to calculate prediction error and updates model parameters using gradient descent methods, such as stochastic gradient descent or an adaptive gradient algorithm, thereby adjusting the weight matrices of the attention and feed-forward layers.

The server uses configuration parameters such as temperature, top-k sampling, and maximum output length to control generative behavior of the model. The generative AI model, when invoked during inference, receives the prompt sentence tokens, computes attention over prior tokens, and generates response text token by token. The server receives the response text as a character string and records it in a log table for traceability. The server then parses the response text. In one embodiment, the server instructs the generative AI model via the prompt to use a specific textual structure (for example, “Recipe 1: . . . Ingredients: . . . Steps: . . .”) so that the server can parse the output using rule-based regular expressions and token segmentation. The server extracts recipe titles, ingredient lists, quantities, and step-by-step cooking procedures from the response text to form cooking proposal information.

The server treats the cooking proposal information as semi-structured data. The server transforms the extracted information into structured records matching the schema of the recipe and ingredient tables, using mapping rules stored in a configuration table. The server then invokes a nutrition value calculation program to recompute energy and nutritional indices for each AI-proposed recipe. The server expands each AI ingredient phrase into canonical ingredient identifiers using dictionary lookup and approximate string matching, and obtains base nutritional composition from the nutritional composition table. The server calculates total energy and nutrient amounts for each recipe by summing ingredient-level contributions based on quantities. The server compares the recalculated indices with thresholds determined from the personal information and health condition information. If a recipe exceeds a limit, such as a maximum sodium intake per meal, the server marks the recipe as violating a constraint and assigns a penalty to its ranking, or excludes it entirely.

The server combines the AI-generated cooking proposal information with the candidate cooking information derived from the database. The server uses a unification procedure that aligns AI proposals with database records where possible. For example, the server may map an AI-proposed “grilled chicken salad” to an existing database recipe if the title similarity and ingredient overlap exceed a threshold. In that case, the server enriches the existing record with additional instructions or explanations from the AI response. If an AI-proposed recipe does not match any existing record, the server stores it as a new virtual recipe entry in a dedicated table, with references to generic ingredient entries. The server then applies an integrated ranking algorithm, which considers both the original composite scores and new relevance scores derived from AI-generated narrative content, such as emphasis on user-specific goals.

The server converts the integrated cooking proposal information into a structured data format suitable for transmission to the terminal. The server may use structured formats to represent lists of recipes, ingredient groups, step sequences, and nutritional tables. The terminal receives this structured information and converts it into display information. The terminal renders the display information on the display device, for example by showing lists of recommended dishes, detailed views of recipes, and visual indicators for constraints such as “low sodium” or “within budget.”

The server also generates interactive prompt sentences for question-and-answer interactions. The user, through the terminal, may input a natural language question such as, “I do not have chicken; what can I use instead for this recipe?” The terminal transmits this question along with context identifiers such as recipe identifiers and user profile identifiers. The server then constructs an interactive prompt sentence that embeds the user question and relevant structured context, such as:
“User profile: low-sodium diet, beginner cooking skill. Current recipe: grilled chicken salad with seasonal vegetables. Ingredients: chicken breast, lettuce, tomato, cucumber, olive oil, lemon juice. User question: ‘I do not have chicken; what can I use instead for this recipe?’ Please propose appropriate substitute ingredients that maintain similar protein content and remain low-sodium, and explain how to adjust cooking steps.”

The server transmits this interactive prompt sentence to the generative AI model. The generative AI model returns answer information including substitute ingredient proposals, such as tofu or white fish, and adjusted cooking steps. The server parses the answer information, maps substitute ingredients to canonical ingredient identifiers, and recomputes nutritional indices for the alternative options. The server may present multiple substitution options along with corresponding nutritional impacts to the user. The terminal displays these alternatives and highlights the technical constraints satisfied, such as preservation of protein level or reduction of sodium.

The server improves computer technology in several ways. The server reduces communication load with the generative AI model by generating prompt sentences that embed compact, pre-filtered candidate information instead of raw, unfiltered data. This reduces the number of tokens transmitted and the size of model outputs, thereby lowering network bandwidth usage and inference time. The server optimizes memory usage and processing time on the server side by representing recipes and user profiles as feature vectors and by performing constraint checking and ranking with vectorized numerical operations. This is technically distinct from a simple automation of human mental steps; human operators would not practically calculate weighted feature vectors and multi-dimensional nutritional constraints across thousands of recipes in real time.

The server also improves data management by unifying structured database records and unstructured AI-generated text into a normalized schema. The server ensures that AI output is converted into canonical data structures and is stored with traceable links to original prompts and constraints. This architecture reduces redundancy and allows incremental updates of recommendation logic without retraining or re-programming the entire system. Because the server uses explicit integration and verification algorithms, the system achieves lower error rates in constraint violation compared to purely rule-based or purely text-based recommendation engines.

The generative AI model in the system uses a non-conventional processing pipeline relative to simple rule engines. The model generates candidate recipes according to high-dimensional patterns learned from training corpora, rather than a fixed rule set. The server then subjects these model outputs to deterministic, rule-based nutritional and cost computations, using explicit numeric thresholds and multi-objective scoring. This hybrid approach of neural generation followed by machine-executable constraint verification yields higher diversity and personalization of recommendations while maintaining strict adherence to technical constraints. The causality is such that the neural generation provides rich variations and the deterministic verification filters those variations, resulting in improved precision of recommendations without requiring additional human supervision.

In alternative embodiments, the server may employ different neural network architectures for the generative AI model, such as encoder-decoder architectures or recurrent neural networks, provided that the model can accept prompt sentences and output natural language responses. The server may use alternative loss functions, such as a combination of cross-entropy loss and coverage loss, during training to improve instruction-following behavior. The server may apply data augmentation strategies, such as paraphrasing prompts or injecting synthetic ingredient lists, during training to improve robustness of generation in meal-planning contexts. The server may also vary the structure of feature vectors or the form of composite scoring functions, such as using logistic regression or gradient-boosted decision trees on top of base features derived from cooking-related information.

In further embodiments, the server may localize nutritional thresholds according to regional guidelines or may apply hardware acceleration for vectorized nutritional computation using parallel processing units. The terminal may vary in form, for example as a wearable terminal or an in-vehicle terminal, and may display meal recommendations in conjunction with other sensor data, such as physical activity levels, further enhancing the technical integration of data sources. In all such embodiments, the core processing remains that the server acquires personal information and cooking-related information, generates and transmits constrained prompt sentences to a generative AI model, verifies and integrates AI responses using structured computational procedures, and returns normalized, constraint-compliant recommendations to the terminal for display to the user.

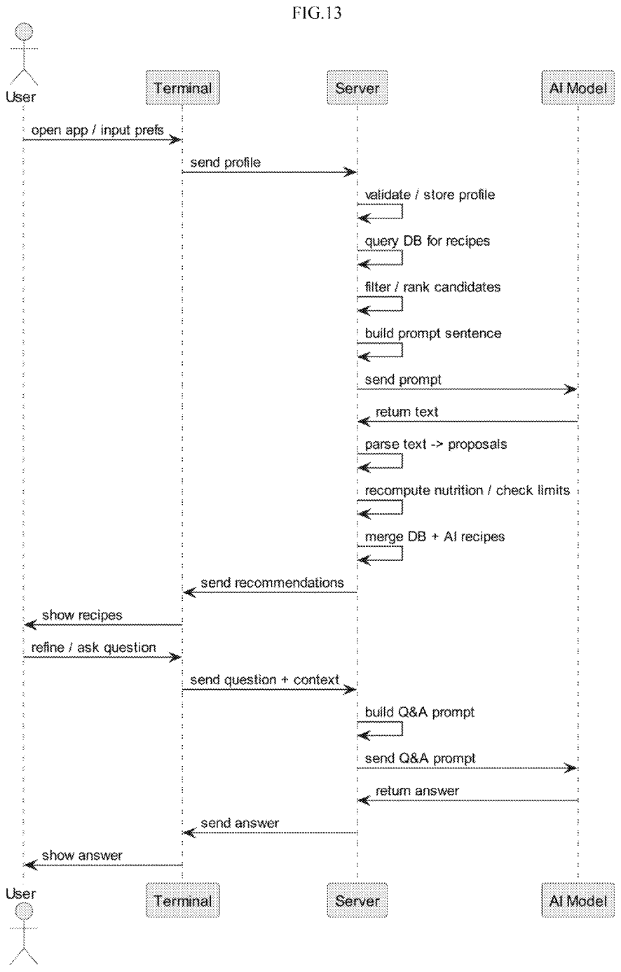

The following describes the processing flow using FIG. 13.

### Step 1:

The user operates the terminal to launch a meal recommendation application.

The terminal displays input fields for budget, health condition, nutritional preference, seasonal preference, cooking skill level, and meal preparation time.

Input: touch or keyboard inputs from the user (raw text, numeric values, selections).

The terminal converts these raw inputs into structured key-value data, for example mapping “daily budget 2,000 JPY” to a numeric field, and mapping “beginner” to a coded skill level.

Output: a structured personal information object stored in the terminal's memory.

### Step 2:

The terminal establishes a secure network connection to the server using HTTPS.

Input: the structured personal information object and a session identifier.

The terminal serializes the personal information into a request payload, attaches authentication or session data to request headers, and transmits the request to a predefined API endpoint of the server.

Output: a network message received by the server containing structured personal information.

### Step 3:

The server receives the network message via a network interface and passes it to an application module.

Input: the serialized personal information and session data.

The server deserializes the payload into internal data structures, validates data types and ranges (for example, checks that the budget is non-negative and that the time is within a configured maximum), and logs the received profile in a logging subsystem.

Output: a validated personal information record stored in server memory and optionally persisted in a user profile table in a storage device.

### Step 4:

The server accesses a storage device to retrieve candidate cooking-related information according to the validated personal information.

Input: the validated personal information record and database schemas for recipes, ingredients, and nutritional values.

The server constructs database queries with constraints derived from the personal information (for example, “estimated_cost<=budget” and “cooking_time<=time_limit”) and executes them using a database management system.

Output: a set of candidate recipe records and associated ingredient and nutritional records loaded into server memory.

### Step 5:

The server transforms the retrieved records into feature vectors suitable for numerical computation.

Input: the candidate recipe records and the corresponding ingredient and nutritional records.

The server calculates normalized values for cost, cooking time, calories, macronutrients, and sodium; the server also derives a skill-level suitability score by comparing required skill tags with the user's skill level.

Output: a list of candidate recipes represented as feature vectors and linked to original record identifiers.

### Step 6:

The server performs filtering and ranking on the feature vectors based on constraints and priorities.

Input: the list of feature vectors and constraint thresholds derived from the personal information.

The server discards vectors that violate hard constraints (for example, cost above budget or time above time limit) and computes a composite score for each remaining vector using a weighted sum or similar scoring function.

Output: an ordered list of candidate cooking information, each with an associated composite score and link to underlying recipe data.

### Step 7:

The server generates a prompt sentence for a generative AI model using the personal information and the ordered candidate cooking information.

Input: the validated personal information and the ordered list of candidate recipes.

The server selects a subset of top-ranked candidates, extracts their titles and key ingredients, and inserts these values into a text template to construct a prompt sentence that describes constraints and candidate options.

Output: a complete prompt sentence in natural language formatted as text data.

### Step 8:

The server transmits the prompt sentence to a generative AI model over a network or internal interface.

Input: the generated prompt sentence and configuration parameters such as temperature and maximum output length.

The server packages the prompt sentence and parameters into an AI request object and sends it to the generative AI model's inference API, then waits for the response.

Output: a response text object returned from the generative AI model containing generated recipe descriptions.

### Step 9:

The server parses the response text returned by the generative AI model into structured cooking proposal information.

Input: the response text and parsing rules or patterns that define expected sections such as recipe names, ingredients, and steps.

The server segments the text, applies pattern matching to identify ingredients and quantities, and maps textual ingredient names to canonical ingredient identifiers stored in the database.

Output: a set of structured cooking proposal records containing recipe names, ingredient lists with quantities, and step-by-step procedures.

### Step 10:

The server recalculates nutritional indices and verifies constraints for each AI-generated cooking proposal.

Input: the structured cooking proposal records and nutritional composition data for mapped ingredients.

The server computes total energy and nutrient amounts per recipe by summing ingredient contributions according to quantities, compares computed values against health and nutritional conditions of the personal information, and flags or removes proposals that exceed limits.

Output: a filtered set of AI-generated cooking proposals with associated nutritional indices and constraint-compliance flags.

### Step 11:

The server integrates the AI-generated cooking proposals with the original candidate cooking information from the database.

Input: the filtered AI-generated cooking proposals and the ordered list of candidate cooking information.

The server determines matches between AI-generated recipes and database recipes based on title similarity and ingredient overlap, merges matched entries by augmenting database records with AI-generated details, and adds unmatched AI recipes as new virtual candidates; the server then computes updated rankings that combine original composite scores and AI-derived relevance measures.

Output: an integrated set of cooking proposal information containing both database-derived and AI-enhanced recipes, each with a final ranking score.

### Step 12:

The server formats the integrated cooking proposal information as structured response data for the terminal.

Input: the integrated set of cooking proposal information and a response schema specifying fields such as title, ingredients, steps, time, cost, and nutritional summary.

The server transforms internal data structures into a structured data format, orders recipes according to final ranking, and attaches metadata such as labels for “AI suggestion” or “low sodium.”

Output: a structured response payload ready for transmission to the terminal.

### Step 13:

The terminal receives the structured response payload from the server and converts it into display information.

Input: the structured response payload and the terminal's display templates.

The terminal parses the payload, populates visual components such as recipe lists and detail views, and renders texts, numbers, and labels on the screen, allowing the user to browse, select, and inspect recommended recipes.

Output: a visual presentation on the display device showing recommended meals and their details.

### Step 14:

The user interacts with the displayed recommendations and may issue additional questions or refinements.

Input: the presented recipes and user inputs such as taps, selections, and natural language questions (for example, substitution or storage questions).

The terminal captures these inputs, packages them with context information such as recipe identifiers, and transmits refinement requests or question data back to the server.

Output: updated structured input to the server that can trigger a new cycle of candidate selection, prompt sentence generation, generative AI interaction, and result integration.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented meal recommendation and ordering systems generally treat user constraints and external context as static filter parameters applied to pre-defined menus. In such systems, a computing device typically retrieves a list of candidate meals from a data storage unit, applies simple rule-based filters based on budget or dietary restrictions, and returns a narrowed list to a user terminal. These approaches suffer from several technical limitations.

First, existing systems do not generate or manage prompt sentences as first-class computational objects for interaction with a generative AI model. In many implementations, the generation of requests to a language model is ad hoc, with limited integration of heterogeneous data sources such as user attribute information, emotion states, weather forecast information, and distribution product information. As a result, the computing device is unable to consistently construct context-rich inputs for the generative AI model, which leads to unstable quality and low personalization of generated outputs.

Second, known systems typically separate recommendation logic from ordering and logistics logic in a loose and manual fashion. A server may display recipes or suggestions to a user, but the mapping from those high-level suggestions to concrete purchasable products, and the generation of machine-readable order data for delivery services, often requires manual user intervention or additional service-specific logic. This separation increases processing latency, computational overhead, and integration complexity in the server, and reduces reliability of automated ordering.

Third, existing systems rarely incorporate user emotion analysis as a dynamic control signal in prompt construction and subsequent recommendation computation. While some applications perform sentiment analysis for user feedback, the emotion data is not systematically fused with contextual information such as environmental conditions (e.g., weather) in a single computational pipeline. Consequently, the server cannot adapt the generative AI model's behavior in real time to prioritize different classes of meal menus (e.g., soothing meals versus celebratory meals) according to user emotion and context, which degrades user satisfaction and reduces effectiveness of personalization.

Fourth, conventional systems generally do not close the loop between user interactions and the generative AI model through machine-readable history information that directly updates both model parameters and prompt generation rules. Recommendation engines may log clicks or purchases, but they do not use unified selection history, preference history, and emotion state information as training signals to refine prompt templates and weighting strategies. This results in static or slowly adapting systems that fail to leverage the full computational potential of machine learning and generative models on modern hardware.

From the viewpoint of computer technology, these limitations manifest as inefficient use of processing resources, increased network and input/output operations between disparate components, and fragile orchestration of AI inference, data retrieval, and ordering operations. The lack of a unified server-side architecture that (i) constructs and modifies prompt sentences based on multi-dimensional context, (ii) parses generative AI outputs into structured data, (iii) automatically maps such data to purchasable goods and order formats, and (iv) continuously updates its own behavior based on accumulated interaction histories, leads to suboptimal system performance, increased latency, and reduced scalability.

Accordingly, there is a need for an improved computer-implemented system that centrally manages prompt sentence generation, generative AI model invocation, output parsing, product mapping, ordering, and feedback-driven adaptation within a single integrated processing pipeline. Such a system should technically enhance the way a server utilizes computation and memory resources to orchestrate heterogeneous data sources, emotion recognition, and generative AI inference, thereby improving throughput, responsiveness, and quality of interactive meal recommendation and ordering services.

The specific processing by the specific processing unit 290 of the data processing device 12 in Application Example 2 is realized by the following means.

The present invention provides a server comprising a processor, a memory, and one or more communication interfaces, the processor being configured to store, in the memory, user attribute information, weather forecast information, cooking procedure information, and distribution product information as training data for a machine learning processing system; to cause the machine learning processing system, including a generative AI model, to learn patterns from the user attribute information, the weather forecast information, the cooking procedure information, and the distribution product information; to receive, via the communication interfaces from a terminal device, user input information including at least one of a natural language query, a budget condition, a health condition, a nutritional condition, a cooking skill level, and a meal preparation time; to integrate the user input information with the user attribute information, the weather forecast information, the cooking procedure information, and the distribution product information; to generate, by executing prompt construction logic, a context-rich prompt sentence to be input into the generative AI model, the prompt sentence specifying constraints and instructions for generating at least one meal menu, ingredient set, and cooking procedure; to analyze a user emotion state by applying an emotion estimation unit to at least one of the user input information, voice information, and image information received from the terminal device; to dynamically modify a content of the prompt sentence in response to the user emotion state and the weather forecast information so as to cause the generative AI model to preferentially generate a meal menu having at least one of a soothing characteristic and a celebratory characteristic corresponding to the user emotion state and an environmental condition; to input the prompt sentence into the generative AI model and obtain, as a natural language generation result, a generated text including at least one of a meal menu candidate, an ingredient candidate, a cooking procedure, and an explanation; to parse the generated text to extract structured information including the meal menu candidate, the ingredient candidate, and the cooking procedure; to map each ingredient included in the ingredient candidate to one or more purchasable products by referencing the distribution product information, calculate cost information and required time information for each meal menu candidate, and generate purchase candidate information including a list of purchasable products associated with each meal menu candidate; to transmit, to the terminal device, presentation information including the meal menu candidate, the associated purchase candidate information, and the cooking procedure such that the terminal device is caused to display the meal menu candidate and accept user selection; to receive, from the terminal device, selection information indicating at least one selected meal menu candidate or at least one selected product, and generate order data for a delivery service based on the selection information and the purchase candidate information; to transmit the order data to the delivery service via the communication interfaces to instruct execution of an order process so that immediate delivery of food or ingredients associated with the selected meal menu candidate is performed; and to store, in the memory, history information including at least a selection history, a preference history, and the user emotion state, and to update at least one of parameters of the machine learning processing system and rules for generating the prompt sentence based on the history information to improve recommendation performance in a subsequent interaction. This enables the server to implement an integrated and adaptive computational pipeline that efficiently coordinates multi-source data acquisition, emotion-aware prompt sentence generation, generative AI model inference, structured output interpretation, automatic mapping to purchasable products, and real-time ordering, thereby improving overall system responsiveness, scalability, and personalization quality from a computer-technical perspective.

The term “user attribute information” refers to information that characterizes a user, including at least one of demographic characteristics, preference characteristics, dietary restriction characteristics, past interaction history, and purchase history, which is used by the server to personalize generation and recommendation processes.

The term “weather forecast information” refers to information representing predicted environmental conditions for a location and time period, including at least one of temperature, precipitation, humidity, and general weather category, obtained from an external information source and used by the server as contextual input.

The term “cooking procedure information” refers to information describing how to prepare a meal, including at least one of step-by-step instructions, required tools, estimated preparation time, and difficulty indicators associated with a recipe.

The term “distribution product information” refers to information describing items that can be acquired through a commercial distribution network, including at least one of product identifiers, product names, package sizes, prices, availability indicators, and supplier identifiers.

The term “machine learning processing system” refers to a computing subsystem that executes one or more machine learning algorithms to learn patterns from training data and to perform inference, the subsystem including at least one trained model stored in a memory and executed by a processor.

The term “generative AI model” refers to a machine learning model configured to generate output data, such as natural language text, in response to input data, by probabilistically producing new content that is not limited to a fixed set of predefined responses.

The term “user input information” refers to information received from a terminal device and originating from a user, including at least one of a natural language query, constraint parameters, selection commands, and control commands, which is used as input for processing by the server.

The term “prompt sentence” refers to a machine-generated or machine-modified sequence of symbols including natural language text and optionally structured tokens, which is constructed by the server and supplied to the generative AI model as an instruction and context for controlling the generation of output by the generative AI model.

The term “emotion state” refers to a computational representation of a psychological condition of a user, including at least one of a classified category such as joy, sadness, fatigue, or stress, and an associated intensity value.

The term “emotion estimation unit” refers to a processing component configured to analyze input signals, including at least one of text, audio, and image data, and to output an estimated emotion state of a user.

The term “terminal device” refers to an information processing apparatus operated directly or indirectly by a user, including at least one of a portable communication device, a stationary computing device, and a display-equipped appliance, which is configured to exchange information with the server.

The term “natural language generation result” refers to output data produced by the generative AI model in response to a prompt sentence, the output data including at least one sequence of natural language tokens and optionally embedded structured elements.

The term “meal menu candidate” refers to a proposed combination of food items and associated preparation information generated or derived by the server, which is considered as a possible option for a user's meal.

The term “ingredient candidate” refers to an item or set of items representing food components required to prepare at least part of a meal menu candidate, as identified or generated by the server.

The term “purchase candidate information” refers to structured information that associates one or more ingredients with one or more purchasable products, including at least product identifiers, prices, and quantities, which is prepared for potential ordering.

The term “presentation information” refers to information transmitted from the server to the terminal device for display or other user-perceivable output, including at least one of meal menu candidates, ingredient lists, cooking procedures, and cost summaries.

The term “selection information” refers to information indicating a choice or preference expressed by a user via the terminal device, including at least one of a selected meal menu candidate, a selected product, and a selection of an operation mode.

The term “order data” refers to machine-readable information that specifies at least one item to be supplied by a delivery service, including at least one of product identifiers, quantities, destination information, and payment-related parameters, and which is suitable for initiating an automated order process.

The term “delivery service” refers to a service system configured to receive order data and to manage physical distribution of goods or prepared food items to a specified destination.

The term “history information” refers to information accumulated over time regarding interactions between the user and the system, including at least selection history, preference history, and associated emotion states, which is used to adapt subsequent processing.

The term “expense constraint information” refers to information specifying a limitation on monetary expenditure acceptable to a user for at least one meal or order, expressed as a value or range.

The term “health state information” refers to information about a physical or medical condition of a user relevant to food intake, including at least allergies, chronic diseases, and dietary restrictions.

The term “nutritional index information” refers to information representing nutritional characteristics, including at least energy, macronutrient content, and micronutrient content, used as criteria for evaluating or constraining meal menus.

The term “ingredient seasonality information” refers to information indicating a preferred or typical time period when an ingredient is considered optimal based on at least availability, cost, or quality.

The term “cooking skill level information” refers to information representing an estimated degree of cooking proficiency of a user, including at least discrete levels such as beginner, intermediate, and advanced.

The term “meal preparation time information” refers to information specifying a time duration available or acceptable to a user for preparing a meal, used as a constraint in menu generation.

In one embodiment, a server, a terminal, and a user cooperate to implement the claimed system. The server includes at least one processor, a memory, a storage device, and one or more network interfaces. The terminal includes at least one processor, a memory, a display unit, an input unit, a microphone, and optionally a camera, and communicates with the server via a communication network such as the Internet.

The server executes an operating system such as a server-class operating system, and executes application software written, for example, in a high-level programming language. The server uses a relational database management system such as a relational data store to store user attribute information, weather forecast information, cooking procedure information, distribution product information, and history information. The server uses a machine learning framework such as a numerical computation framework or a neural network framework to implement a machine learning processing system including a generative AI model and an emotion estimation unit.

The server stores user attribute information in the relational database in a structured format. The server represents a user by a user identifier and stores, for each user, at least allergy attributes, preference attributes (for example, preferred ingredients, disliked ingredients), health state attributes, cooking skill level attributes, and past interaction history. The server also stores expense constraint information and meal preparation time information in tables associated with user identifiers. The server uses structured records with columns for numeric attributes (for example, maximum budget per meal, available time in minutes) and categorical attributes (for example, beginner, intermediate, advanced).

The server stores cooking procedure information as recipe records in the relational database. Each recipe record includes at least a recipe identifier, recipe title, ingredient list, step-by-step instructions, estimated preparation time, difficulty level, and nutritional index information such as calories, macronutrient values, and micronutrient values. The server represents ingredient lists as a set of ingredient records with ingredient names, quantities, units, and optional tags (for example, “spicy,” “warming,” “light”).

The server stores distribution product information as product records in the relational database or a separate product database. Each product record includes at least a product identifier, product name, associated ingredient name, package size, price, availability flag, supplier identifier, and estimated delivery time. The server optionally caches distribution product information obtained from external shopping APIs.

The server acquires weather forecast information from an external weather service over the network. The server sends a request including geographic coordinates or a location identifier, and receives, as a response, forecast data including temperature, precipitation probability, humidity, and general weather description. The server stores this weather forecast information in the database or a short-term cache and annotates each record with time stamps and locations. The server converts continuous numeric values such as temperature into categories (for example, cold, moderate, hot) using predetermined thresholds, thereby generating features that are later provided to the machine learning processing system.

The server implements a machine learning processing system using a neural network framework. In one embodiment, the server constructs a generative AI model as a transformer-based neural network with multiple self-attention layers, feed-forward layers, and embedding layers. The server uses a vocabulary-based tokenizer to convert prompt sentences into sequences of tokens. The server embeds each token into a continuous vector space and processes the sequence through the transformer blocks. The server obtains, for each output token position, a probability distribution over the vocabulary and selects tokens according to a sampling strategy such as greedy decoding or top-k sampling.

The server trains the generative AI model in advance using a training data set that includes past prompt sentences and corresponding desired outputs such as meal menu descriptions and ingredient lists. The server uses a loss function such as cross-entropy loss between predicted token probabilities and reference token indexes. The server updates model weights by performing gradient descent or a variant such as Adam optimization. The server optionally performs fine-tuning using domain-specific data that includes structured user context (for example, budget, emotion, weather) serialized into textual form. The server can perform training offline on a separate training machine, and then deploy the trained model parameters onto the inference server.

The server further implements an emotion estimation unit. In one embodiment, the server uses a neural network classifier based on a bidirectional recurrent neural network or a transformer encoder model. The server receives text input, audio-derived features, or image-derived features and converts them into features such as word embeddings, Mel-frequency cepstral coefficients (MFCCs), or facial landmark coordinates. The server feeds the features into the classifier and obtains a probability distribution over a set of emotion categories such as joy, sadness, fatigue, stress, and neutrality. The server uses a softmax output layer and cross-entropy loss during training, and updates classifier weights using gradient-based learning. The server stores, in the memory, configuration parameters defining thresholds for selecting the dominant emotion and for ignoring low-confidence emotion estimates.

The terminal runs a client application that presents a graphical user interface. The terminal displays input fields for a budget value, a meal preparation time, a health goal, a cooking skill level, and free-form natural language text. The terminal also provides buttons or toggles for specifying whether the user wants to cook personally, receive ingredients, or receive prepared meals. The terminal captures user voice via the microphone and optionally captures user facial images via the camera. The terminal encodes the captured signals and transmits them to the server over the network.

In one mode, the terminal sends raw audio or images to the server. In another mode, the terminal performs local pre-processing using a local library such as a speech recognition engine or a facial expression detector, and transmits text or pre-computed emotion features to the server. The terminal receives presentation information from the server and displays meal menu candidates, ingredient lists, cooking procedures, and cost information in a structured layout. The terminal highlights attributes such as “Beginner,” “Under 20 minutes,” “Comforting for a cold day,” according to metadata included in the presentation information.

The server constructs prompt sentences for the generative AI model in a specific way that differs from conventional rule-based recommendation logic. The server does not merely pass raw user queries to the model. Instead, the server integrates multiple heterogeneous data sources and converts them into a unified textual representation. The server includes, in the prompt sentence, explicit sections describing user attribute information, emotion state, weather forecast information, recipe search constraints, product mapping constraints, and output formatting instructions.

For example, the server can generate a prompt sentence in the following form: “You are a generative AI model that suggests personalized meal plans.

User profile:
Allergies: no peanuts, no shrimp.
Preferences: likes chicken, dislikes lamb.
Health goal: low fat, high protein.
Cooking skill: beginner.

Current context:
Emotion: tired.
Weather: hot and humid.
Budget: 1,000 yen total.
Preparation time: 20 minutes or less.

### Task:

Suggest two dinner recipes that fit all conditions above.

For each recipe, output:
1) Recipe name,
2) Main ingredients with approximate quantities,
3) Simple step-by-step instructions in three to five steps,
4) Short explanation why this recipe matches the user's emotion and weather.”

In another example, when the server wants to convert a selected recipe into a structured ingredient list, the server can generate a prompt sentence such as:
“From the recipe ‘cold chicken soba salad’, extract an ingredient list with quantities suitable for one adult. Output the ingredient names and approximate quantities in plain text form.”

In yet another example, when the server receives a free-form question from the user, the server can generate a prompt sentence such as:
“Considering the user's past meal history and today's cool, rainy weather, and the user's current emotion (sad), answer the following question: ‘I don't know what to make for dinner today.’ Propose two warm, comforting dinner recipes that can be cooked in under 30 minutes. Provide ingredient lists and simple instructions.”

The server generates these prompt sentences by applying prompt construction logic implemented in program code. The server retrieves user attribute information and history information from the database using structured queries. The server converts categorical attributes and numeric attributes into descriptive phrases by referencing template strings stored in the memory. The server obtains weather categories from the weather forecast information and converts them into textual descriptions such as “hot and humid” or “cold and rainy.” The server obtains emotion state from the emotion estimation unit and converts it into terms such as “tired,” “sad,” or “happy.” The server then concatenates these textual segments according to predetermined template structures, while inserting constraints (such as budget and time) and explicit output format instructions.

The server applies a non-conventional rule for dynamically modifying prompt sentences in response to emotion state and environmental conditions. The server maintains, in the memory, a mapping between emotion categories and preference weights for certain recipe tags. For example, the server maintains that when the emotion state is “sad,” recipes tagged as “warm” and “comforting” receive higher selection weights. The server does not rely only on user-expressed preferences but uses the emotion state as a control signal that adjusts how the generative AI model is instructed. When the server constructs the prompt sentence, the server inserts phrases such as “warm, comforting dinner recipes” when sadness is detected, or phrases such as “light, refreshing dishes” when the user is tired and weather is hot. This rule-based insertion of emotion-and weather-dependent descriptions is performed before generative inference. As a result, the generative AI model receives more precise, technically structured guidance, which leads to more relevant and stable output.

The server parses the generated text returned by the generative AI model using deterministic parsing algorithms. The server identifies headings indicating different recipes, such as “Recipe 1” and “Recipe 2,” and splits the text accordingly. The server uses pattern matching to extract list structures from lines starting with markers (for example, hyphens or numerals) and identifies them as ingredient entries or cooking steps. The server builds internal data structures in memory, such as arrays or lists of ingredients with names and quantities, and arrays of steps as ordered strings. The server also extracts short explanation sentences that indicate why a recipe matches the user's context.

The server maps ingredients to purchasable products by querying the distribution product information. The server performs fuzzy matching between ingredient names and product names, using string similarity measures or pre-defined mapping tables. The server also applies filters on product availability and price. For each ingredient, the server selects a product whose name, size, and price best fit the required quantity and the user's budget constraints. The server then calculates an estimated total cost per recipe by summing product prices proportionally to the required quantities. The server calculates an expected preparation time by combining the recipe's estimated preparation time from cooking procedure information and additional time due to differences in product packaging (for example, pre-cut vegetables reduce time).

The server generates purchase candidate information by associating each ingredient with one or more product identifiers and including cost and expected delivery time information. The server transmits, to the terminal, presentation information that contains structured data and textual summaries. The terminal renders these data into user-friendly screens that show meal menu candidates along with “Order ingredients” or “Order prepared meal” options.

When the user selects a meal menu candidate or a specific product on the terminal, the terminal transmits selection information to the server. The server retrieves the corresponding purchase candidate information and forms order data suitable for a delivery service interface. The server converts internal product identifiers into delivery service-specific item codes and includes address and payment parameters stored in the user attribute information. The server transmits the order data via an API of a delivery service. The delivery service then executes an order process and initiates physical delivery of ingredients or prepared meals.

The server stores, in the memory and storage device, history information describing which meal menu candidates were shown, which were selected, which were rejected, and what emotion state was detected at those times. The server aggregates such history information and uses it to adjust model parameters and prompt generation rules. In one embodiment, the server periodically performs fine-tuning of the generative AI model using newly collected prompt-output-feedback tuples. The server treats user selections as positive examples and user rejections as negative examples, and adjusts the training data distribution accordingly. The server also adjusts template parameters for prompt construction, such as the relative emphasis placed on budget constraints versus emotion-based descriptors.

By implementing this architecture, the server improves computer technology in several ways. The server reduces communication overhead and latency by converting a large amount of heterogeneous structured data (user attribute information, weather data, product catalog data, and emotion state) into a single compact prompt sentence instead of issuing multiple separate AI queries or manually orchestrated rule-based operations. The server improves computational efficiency because the generative AI model operates on a context-rich representation that minimizes the need for repeated retrieval and recomputation.

The server improves recommendation accuracy because it embeds multi-dimensional constraints and emotion state into the prompt sentence in a consistent, machine-controlled way. The server does not simply automate human expert behavior; instead, the server introduces a machine-specific representation (the prompt sentence with explicit sections and constraints) that leverages the capabilities of a transformer-based generative model to perform multi-criteria reasoning under constraints that would be difficult and time-consuming for a human to apply consistently.

The server improves data management because it normalizes various data types into specific data structures (for example, tabular user data, recipe graphs, product catalogs) and uses well-defined conversion rules to serialize these structures into prompt sentences and back into structured results. This reduces ambiguity and error rates when mapping AI-generated outputs to real-world items, and facilitates systematic logging and retraining.

The server improves processing speed and reduces error by employing a dedicated emotion estimation unit rather than leaving emotional context to implicit interpretation by the generative AI model. By providing explicit emotion state to the prompt sentence, the server prevents the model from misinterpreting user mood and reduces the number of iterations necessary to achieve satisfactory recommendations.

The system is not limited to a single implementation. In another embodiment, the server can deploy the generative AI model on a dedicated hardware accelerator, such as a specialized neural network accelerator, to further increase inference speed. In another embodiment, the server can use different neural network architectures, such as a mixture-of-experts transformer, to reduce the computational cost for each inference while maintaining quality. In yet another embodiment, the terminal can perform partial inference of the emotion estimation unit locally to reduce server-side computation and network traffic.

In all these embodiments, the server, the terminal, and the user cooperate through clearly defined data structures and processing modules so that the generative AI model and prompt sentence mechanism are used to coordinate and control real-world technical operations: selection, pricing, and ordering of physical goods and preparation of meals, with improved computational efficiency, accuracy, and adaptability compared to conventional systems.

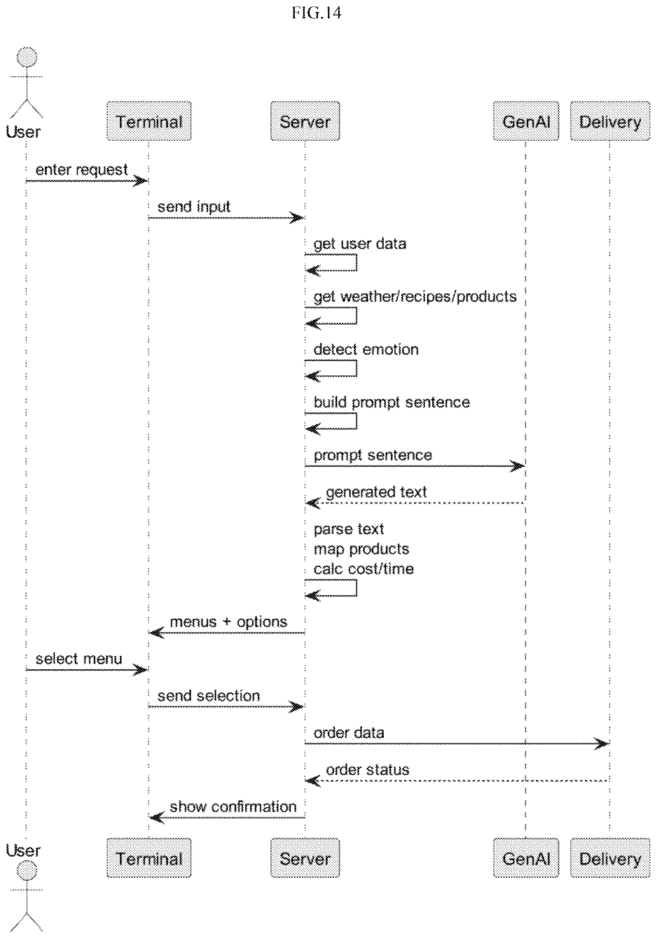

The following describes the processing flow using FIG. 14.

### Step 1:

User inputs conditions and questions on the terminal.

User operates the terminal to enter at least one of a natural language query (for example, “I don't know what to make for dinner today”), a budget value, a desired meal preparation time, a cooking skill level, and a health goal. User optionally allows the microphone and camera of the terminal to be used.

Input: User's keystrokes, touch operations, voice, and facial expressions.

Output: Structured user input information consisting of text strings, numeric values (for example, budget, time), and raw audio/video streams.

Terminal converts touch and keyboard events into text fields, records numeric fields as typed, and buffers audio and video frames in memory.

### Step 2:

Terminal preprocesses user signals and sends a request to the server.

Terminal optionally performs speech-to-text conversion on captured audio using a local speech recognition library and may detect coarse emotion features from facial images. Terminal packages the free-form query text, structured numeric conditions, user identifier, and any locally derived emotion features into a request payload. Terminal establishes an encrypted communication channel to the server and transmits the payload as a structured message.

Input: Raw user input information from Step 1 (text, numbers, audio, images).

Output: A request message including user input information, user identifier, and optional precomputed emotion features, encoded as a structured data object.

Terminal performs data processing by normalizing numeric units (for example, converting budget to a standard currency), encoding text in a standard character encoding, and compressing or encoding audio/video if included, then sends the processed payload to the server.

### Step 3:

Server receives and parses the request message.

Server listens on a network interface and receives the request from the terminal. Server decodes the structured data object and extracts fields such as user identifier, query text, budget value, preparation time, and any preliminary emotion features. Server validates the data types and ranges, and logs the request for monitoring and debugging.

Input: Structured request message from the terminal.

Output: Internal request context object stored in memory, containing normalized user input fields.

Server performs data parsing and validation operations, such as JSON or similar decoding, type checks, and default-value substitution where inputs are missing.

### Step 4:

Server retrieves user attribute information and history.

Server uses the user identifier from the request context to query the relational database for user attribute information, including allergies, preferences, health state, cooking skill level, stored budget preferences, and past interaction history. Server also retrieves selection history and preference history to build a richer profile.

Input: User identifier from the request context.

Output: User attribute information object and history information object.

Server performs data access operations using database queries, joins multiple tables, and constructs a combined in-memory representation, converting categorical attributes to internal codes and numeric values to normalized ranges.

### Step 5:

Server acquires weather forecast information and relevant context.

Server obtains location information (for example, from user attribute information or from the request). Server calls an external weather information service via the network and receives forecast data for the relevant time window. Server converts raw numeric values such as temperature and precipitation into categorical features such as “cold,” “hot,” or “rainy” according to predefined thresholds.

Input: Location information and time context extracted from the request and user attribute information.

Output: Weather forecast information object with both raw values and derived categorical features.

Server performs network I/O operations to call an external API, then applies computational operations such as threshold comparison and mapping to descriptive labels.

### Step 6:

Server estimates the user's emotion state.

Server combines any emotion features sent from the terminal with the user's query text and optionally derived features from audio or image data. Server feeds these features into an emotion estimation unit, implemented as a neural network classifier or similar model, that outputs probabilities for each emotion category. Server selects the dominant emotion based on maximum probability and confidence thresholds.

Input: User query text, optional audio-derived features, optional image-derived features, and optional terminal-side emotion hints.

Output: Emotion state object including an emotion category (for example, “tired,” “sad,” “happy”) and an intensity value.

Server performs feature extraction operations (for example, tokenization, vectorization) and inference operations (matrix multiplications, activation functions) in the emotion estimation model to compute the emotion distribution and selects the final emotion category.

### Step 7:

Server collects recipe and product candidates.

Server queries the recipe database for recipes that are approximately compatible with user constraints such as maximum preparation time and cooking skill level. Server also retrieves nutritional index information and any tags such as “warm,” “refreshing,” or “celebratory.” Server queries distribution product information to obtain lists of purchasable items associated with ingredients typically used in those recipes.

Input: User constraints (budget, time, skill, health goals) and user attribute information from Step 4.

Output: Preliminary recipe candidate set and related product candidate set.

Server performs database search and filtering operations using constraints as conditions, then prepares intermediate sets of candidate recipes and products in memory for later refinement.

### Step 8:

Server constructs a context-rich prompt sentence for the generative AI model.

Server integrates user attribute information, weather forecast information, emotion state, preliminary recipe candidates, and user constraints. Server then generates a prompt sentence by inserting these elements into predetermined templates. The prompt sentence explicitly instructs the generative AI model to produce a set of meal menu candidates with ingredients and simple step-by-step cooking instructions, taking into account budget, time, emotion, and weather.

Input: User attribute information, weather forecast information, emotion state, user input information, and preliminary candidate sets.

Output: A prompt sentence string to be supplied to the generative AI model.

Server performs text-generation operations using template substitution logic, concatenating text fragments, converting numeric and categorical features to descriptive phrases, and embedding explicit instructions (for example, “Suggest two dinner recipes that fit all conditions above.”).

### Step 9:

Server dynamically adjusts the prompt sentence based on emotion and environment.

Server applies stored rules that map emotion categories and weather categories to preferred recipe tags (for example, “warm,” “comforting,” or “light”). Server modifies the prompt sentence by inserting or replacing descriptive phrases so that the generative AI model is guided to prioritize recipes that match the user's emotional and environmental context.

Input: Initial prompt sentence and rules relating emotion state and weather categories to recipe tag preferences.

Output: Adjusted prompt sentence with emotion-and weather-aware instructions.

Server performs rule-based text modification, such as conditional insertion of phrases like “warm, comforting dinner recipes” when the emotion state is “sad” and weather is “cold and rainy,” using pattern matching and string concatenation.

### Step 10:

Server executes generative inference using the generative AI model.

Server tokenizes the adjusted prompt sentence and converts tokens into numerical vectors using learned embeddings. Server feeds the token sequence into a transformer-based generative AI model deployed locally or accessed via an external AI service. The model processes the sequence with self-attention and feed-forward layers to compute distributions over possible next tokens and sequentially generates the response text.

Input: Adjusted prompt sentence string.

Output: Generated text containing one or more meal menu candidates, ingredient descriptions, cooking procedures, and contextual explanations.

Server performs numerical operations including matrix multiplications, attention weight calculations, and activation functions across multiple neural network layers, then decodes the output token sequence into a human-readable text string.

### Step 11:

Server parses the generated text into structured menu data.

Server analyzes the generated text and splits it into individual recipes using section markers or headings. Server detects ingredient sections and step-by-step instruction lines using pattern recognition (for example, identifying lists or numbered lines). Server constructs internal structured objects containing, for each recipe, a recipe title, an ingredient list with approximate quantities, and a sequence of cooking steps.

Input: Generated text from the generative AI model.

Output: Structured menu data objects representing meal menu candidates.

Server performs string processing operations, such as line splitting, regular expression matching, and pattern-based segmentation, to convert free-form text into structured in-memory data.

### Step 12:

Server maps ingredients to purchasable products and calculates costs and times.

Server takes each ingredient entry in the structured menu data and matches it against distribution product information using ingredient names and tags. Server uses string similarity, alias tables, and product tags to identify suitable products. Server then calculates an estimated cost per menu by combining product prices, accounting for package sizes and required quantities. Server also refines expected preparation time estimates based on recipe details and product types (for example, pre-cut versus whole ingredients).

Input: Structured menu data from Step 11 and distribution product information from Step 7.

Output: Purchase candidate information containing product mappings, cost estimates, and refined preparation time for each menu.

Server performs data join operations, numerical calculations for quantity scaling and cost summation, and applies heuristic rules to adjust time estimates depending on product form.

### Step 13:

Server generates presentation information and sends it to the terminal.

Server composes presentation information that includes, for each meal menu candidate, the recipe name, short description, ingredient list, mapped products with prices, and cooking steps, along with total estimated cost and preparation time. Server also includes metadata indicating why each recipe is appropriate given the user's emotion and weather context. The server transmits this presentation information to the terminal over the network.

Input: Structured menu data and purchase candidate information.

Output: Presentation information object transmitted to the terminal.

Server performs data serialization, formatting, and network transmission operations, converting internal objects into a structured format suitable for efficient rendering by the terminal.

### Step 14:

Terminal displays meal menus and accepts user selection.

Terminal receives the presentation information and parses it. Terminal renders multiple meal menu candidates on the display, showing names, costs, preparation times, and high-level explanations. Terminal provides interactive elements such as buttons for “Order ingredients,” “Order prepared meal,” or “Show another suggestion.”

Input: Presentation information from the server.

Output: Visual and interactive user interface elements and, upon interaction, selection information.

Terminal performs UI layout operations, maps structured fields to display components, and monitors user input events linked to specific meal menu candidates or operation modes.

### Step 15:

User selects a meal menu or modifies conditions.

User reviews the proposed menus on the terminal and chooses one of them, or adjusts constraints such as budget, preparation time, or dietary preference. User triggers an action, such as ordering ingredients for a selected menu.

Input: Displayed menu information and user's intention.

Output: Selection information or updated user input conditions captured by the terminal.

Terminal converts user taps or clicks into selection messages and updates stored parameters if the user changes constraints.

### Step 16:

Terminal sends selection information back to the server.

Terminal packages the user's selection, including selected menu identifier and desired ordering mode (ingredients or prepared meal), along with any updated constraints, into a structured message. Terminal sends this message to the server via the network.

Input: User's selection information from Step 15.

Output: Selection request message transmitted to the server.

Terminal performs message construction and network transmission operations, ensuring that identifiers and mode flags are unambiguously encoded.

### Step 17:

Server generates order data and instructs the delivery service.

Server receives the selection request and retrieves the corresponding purchase candidate information for the selected menu. Server forms order data that specifies selected products, quantities, user delivery address, and payment-related parameters. Server converts internal product identifiers into identifiers understood by the designated delivery service and conforms to the service's order format. Server then sends the order data to the delivery service's interface to initiate an order.

Input: Selection request message and purchase candidate information.

Output: Order data transmitted to the delivery service and internal confirmation data.

Server performs data mapping, field transformation, and network communication operations to generate a valid, machine-readable order and to record the outcome.

### Step 18:

Server and terminal handle confirmation and update history.

Server receives confirmation or status information from the delivery service, including order identifier, expected delivery time, and final cost. Server logs this information and updates history information for the user with details such as menu selected, time of selection, prices, and associated emotion state. Server then sends a confirmation message to the terminal. Terminal displays an order confirmation screen showing delivery time and cost.

Input: Delivery service response and previously stored selection context.

Output: Updated history information in the server and confirmation message displayed on the terminal.

Server performs logging and history-writing operations in the database, while the terminal performs UI update operations to present the confirmation to the user.

### Step 19:

Server periodically adapts models and prompt construction rules based on history.

Server analyzes accumulated history information, including which menus were frequently selected under which emotion and weather conditions. Server uses this information to fine-tune internal model parameters and adjust prompt construction rules, for example by changing the weights of certain descriptors or modifying template phrases.

Input: History information aggregated across interactions.

Output: Updated model parameters and updated prompt construction rules stored in memory and storage.

Server performs statistical analysis and machine learning update operations, such as recalculating feature importance, retraining components of the generative AI model or auxiliary models using gradient-based learning, and modifying configuration tables that control how prompt sentences are generated and adjusted in future sessions.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like.

The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

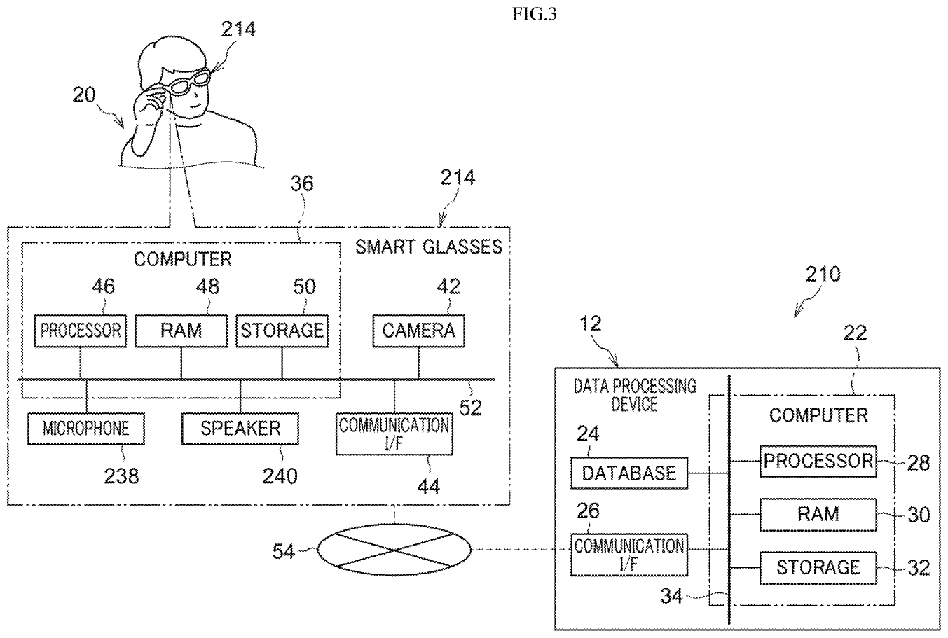

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

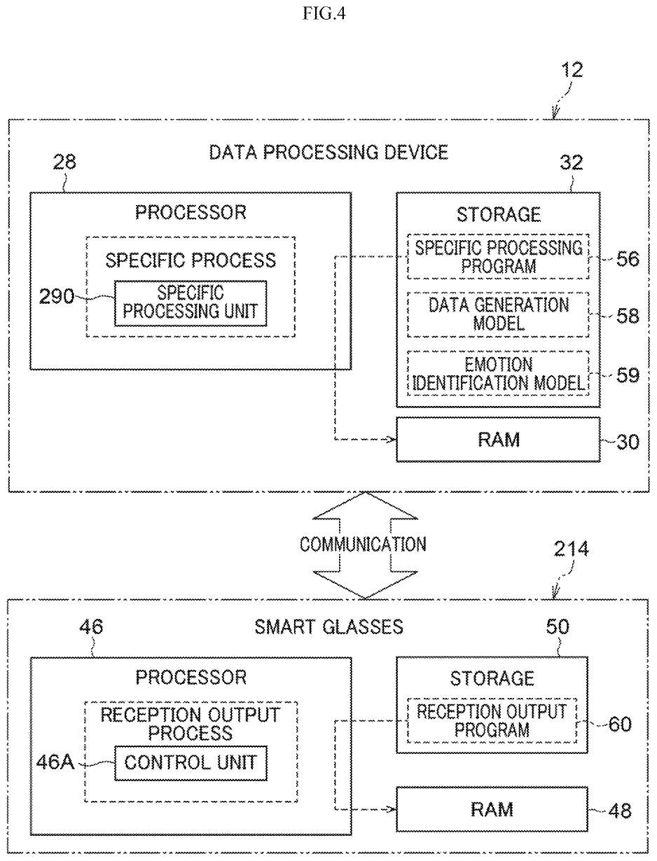

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

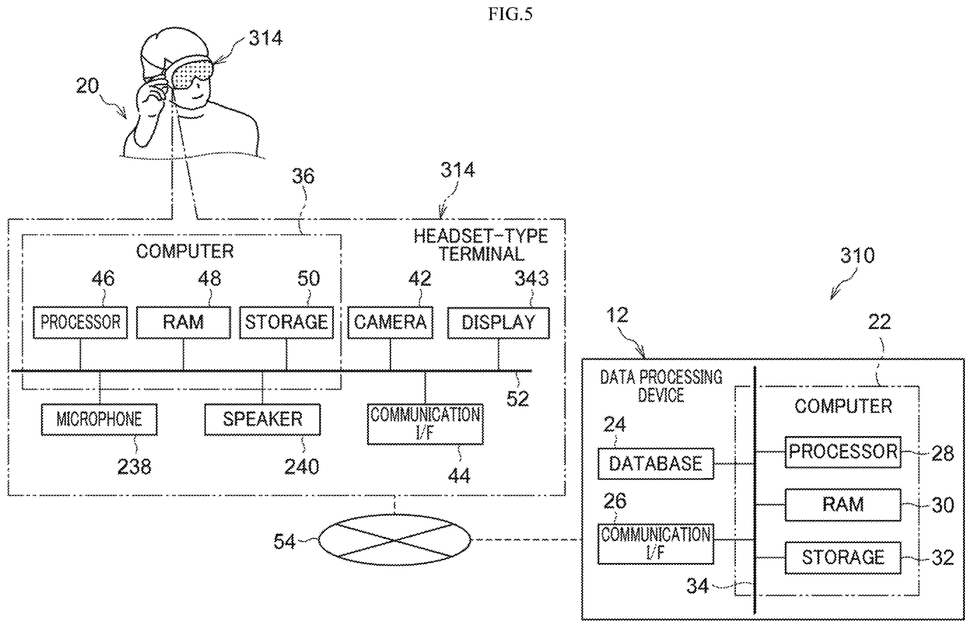

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

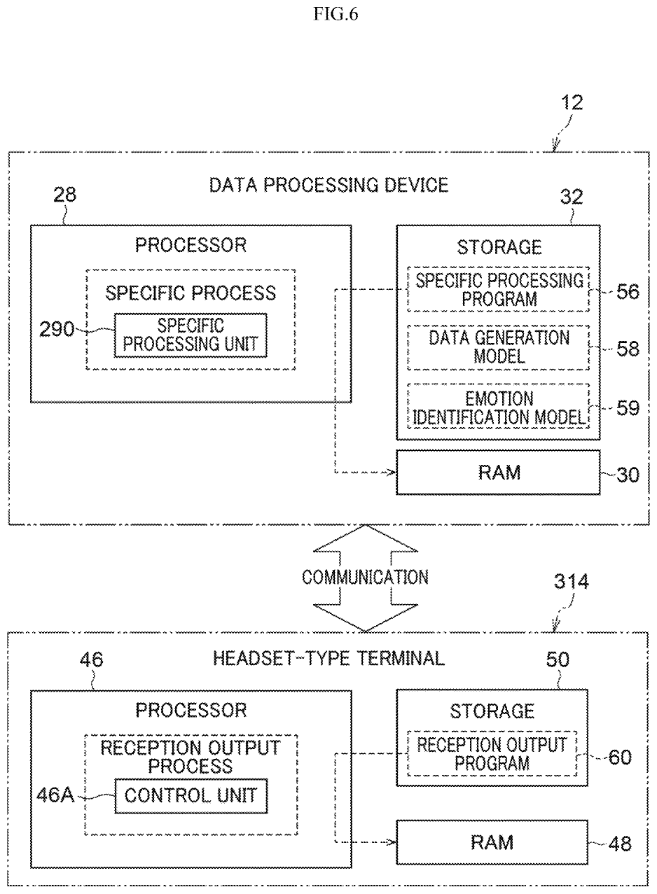

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

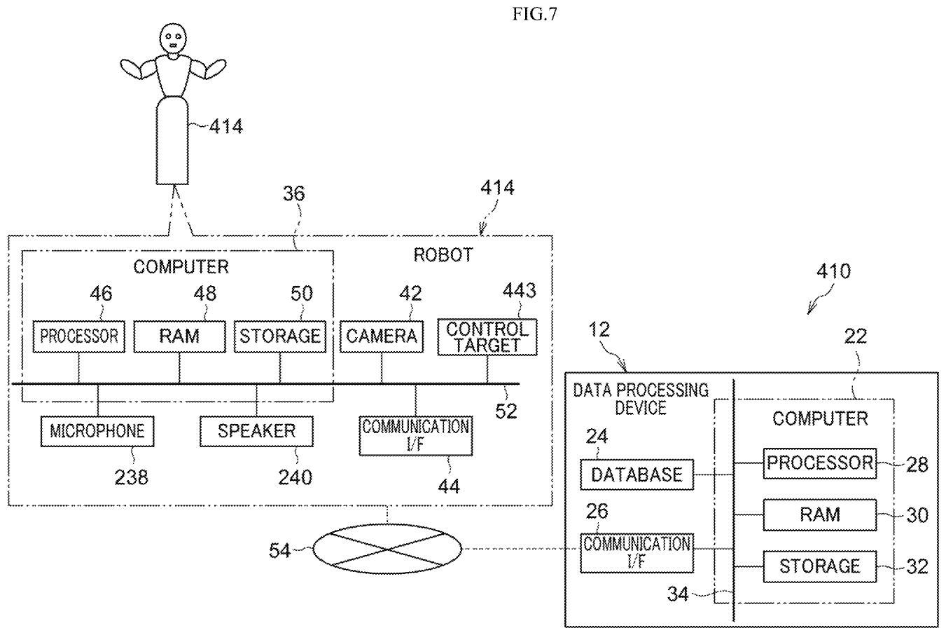

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

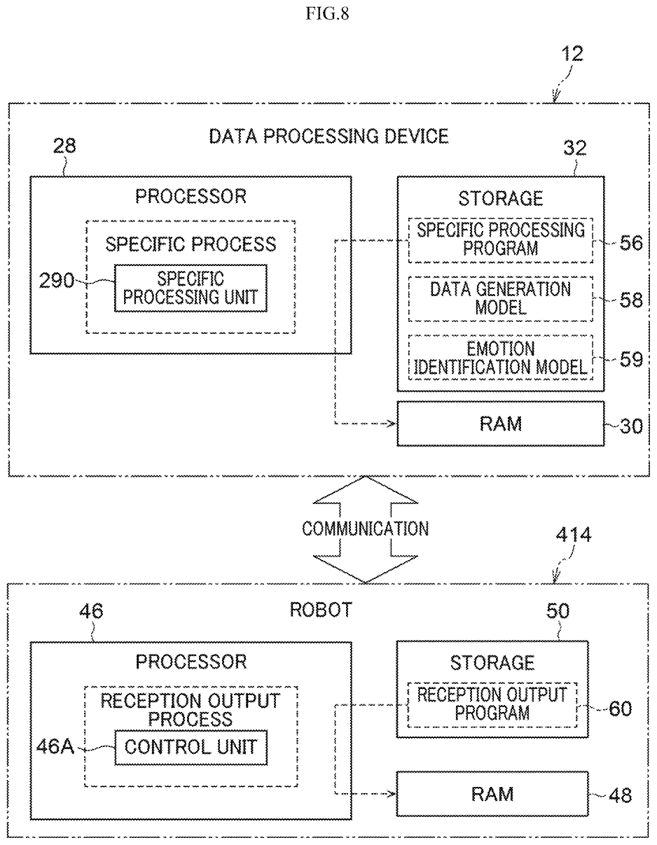

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

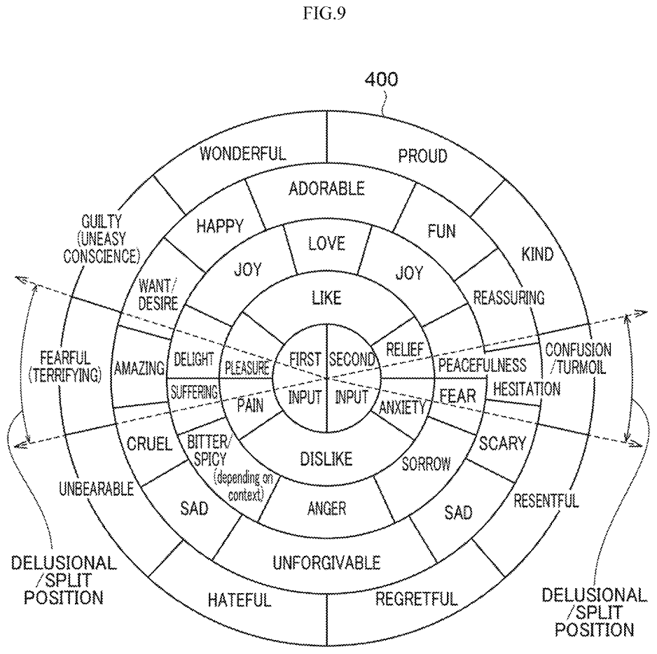

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

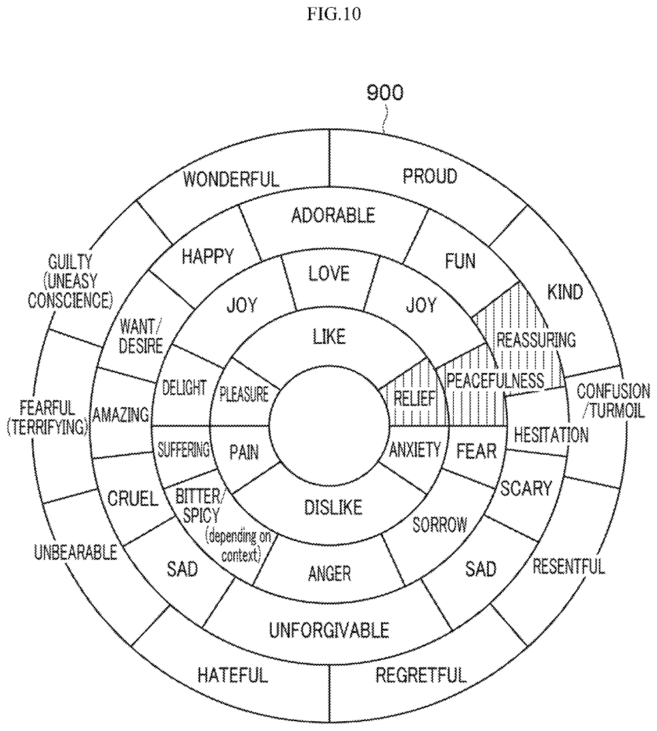

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

Example 1

### (Supplementary 1)

A system comprising a processor,
wherein the processor is configured to
cause a storage unit to store personal data, weather data acquired via an information acquisition unit, cooking information data including cooking procedures, and product information data including sales information, and to cause a generative AI model to learn from the personal data, the weather data, the cooking information data, and the product information data; and
generate a prompt sentence based on the personal data, the weather data, the cooking information data, and the product information data, the prompt sentence being configured to instruct the generative AI model to generate a meal plan adapted to a preference and a health condition of a user and to a weather condition at a current time; and
associate ingredient information included in the meal plan output by the generative AI model with the product information data, specify items purchasable as products that correspond to ingredients required for the meal plan, and generate purchase support information including sales location information and online purchase link information for the specified products; and
control a terminal device to display the meal plan and the purchase support information; and update the personal data and conditions for generating the prompt sentence based on evaluation information or behavior history information acquired from the terminal device, and reflect the updated personal data and the updated conditions in a subsequent meal plan generation process.

### (Supplementary 2)

The system according to supplementary 1,
wherein the processor is configured to
integrate budget information of the user, nutritional conditions, available time, cooking skill level, and information regarding seasonal ingredients with the personal data and the product information data, and generate a prompt sentence that explicitly describes constraint conditions and priority conditions including the budget information, the nutritional conditions, the available time, the cooking skill level, and the information regarding the seasonal ingredients, and instruct the generative AI model to output the meal plan, recipe information, and ingredient candidates that satisfy the constraint conditions and the priority conditions.

### (Supplementary 3)

The system according to supplementary 1,
wherein the processor is configured to
acquire inquiry information regarding food, nutrition, cooking methods, or purchasing methods from the terminal device, generate a prompt sentence including the inquiry information, the personal data, the weather data, the cooking information data, and the product information data, and instruct the generative AI model to generate answer information responsive to the inquiry information and a related meal plan.

### Application Example 1

### (Supplementary 1)

A system comprising a processor,
wherein the processor is configured to
acquire, from a storage unit, personal information including at least user preferences, allergy information, health condition information, budget information, cooking skill information, and lifestyle schedule information, environmental information acquired via a communication network including at least weather information, cooking information including at least cooking procedure information and ingredient information, and product information acquired via a communication network including at least product item information and delivery condition information,
and generate, on the basis of at least the personal information and the environmental information, a prompt sentence to be input to a generative AI model, and cause the generative AI model to generate one or more meal candidates and ingredient information corresponding to the one or more meal candidates,
and transmit, to a terminal device, presentation information including the one or more meal candidates and the ingredient information generated by the generative AI model, and receive, from the terminal device, selection information indicating at least one selected meal candidate among the one or more meal candidates,
and, on the basis of the selection information and the ingredient information, search the product information, and acquire a plurality of product candidates and delivery information corresponding to the ingredient information,
and transmit, to the terminal device, the plurality of product candidates and the delivery information, receive, from the terminal device, order confirmation information, and, on the basis of the order confirmation information, request an external service to execute a purchase process and a delivery process for the plurality of product candidates,
and estimate, on the basis of at least the personal information and a presentation history of the one or more meal candidates, at least one of a user emotional state and a user preference tendency, and generate a prompt sentence including an estimation result, and cause the generative AI model to generate an additional meal candidate corresponding to at least one of the user emotional state and the user preference tendency.

### (Supplementary 2)

The system according to supplementary 1,
wherein the processor is configured to
generate a prompt sentence including at least the budget information, the health condition information, nutritional condition information, seasonality information, the cooking skill information, and meal preparation time information included in the personal information, input the prompt sentence to the generative AI model, and cause the generative AI model to generate an optimal meal candidate including at least cooking procedure information and ingredient information that satisfy conditions represented by the personal information.

### (Supplementary 3)

The system according to supplementary 1,
wherein the processor is configured to
receive, from the terminal device, question information related to at least one of food, nutrition, cooking methods, storage methods, hygiene management, and lifestyle habits, generate a prompt sentence including the question information, input the prompt sentence to the generative AI model, cause the generative AI model to generate response information corresponding to the question information, and transmit the response information to the terminal device.

### Example 2

### (Supplementary 1)

A system comprising a processor and a storage device,
wherein the processor is configured to
acquire personal information regarding a lifestyle of a user via an input apparatus connected to an information processing apparatus, the personal information including at least one of budget information, health condition information, nutritional condition information, seasonal condition information, skill level information, and meal preparation time information, query structured information stored in the storage device based on the personal information and identification information associated with the personal information,
obtain, as cooking-related information, structured information from a data management apparatus, the cooking-related information including at least one of cost information, cooking time information, cooking procedure information, ingredient attribute information, and nutritional component information,
perform numerical operations and logical operations by executing a program stored in a recording medium, the program being configured to apply filtering processing and ranking processing to the cooking-related information in accordance with at least one of a cost condition, a time condition, a nutritional condition, and a user skill condition, and to thereby generate candidate cooking information,
generate a prompt sentence, as text data for input to a generative AI model, based on the personal information and the candidate cooking information,
transmit the prompt sentence to the generative AI model provided externally or internally, receive response text from the generative AI model, parse the response text to extract cooking proposal information, and integrate the cooking proposal information with the candidate cooking information, and
convert integrated cooking proposal information into a structured data format as response data to be transmitted to a user terminal, and output the structured data as display information.

### (Supplementary 2)

The system according to supplementary 1,
wherein the processor is configured to
execute a nutrition value calculation program based on ingredient information and nutritional component information included in the candidate cooking information and the cooking proposal information, recalculate intake energy and a plurality of nutritional indices, and generate final cooking recommendation information by excluding or lowering a ranking of cooking proposal information in which the recalculation result does not satisfy at least one of a health condition and a nutritional condition included in the personal information.

### (Supplementary 3)

The system according to supplementary 1,
wherein the processor is configured to
generate an interactive prompt sentence, as a prompt sentence for dialogue, based on the personal information and the cooking-related information, the interactive prompt sentence using an inquiry sentence regarding food and cooking as input to the generative AI model, supply the interactive prompt sentence to the generative AI model, obtain answer information including at least one of substitute ingredient information, simplified cooking procedure information, storage method information, and safety-related information, and output the answer information as display information to be presented to the user terminal.

### Application Example 2

### (Supplementary 1)

A system comprising a processor,
wherein the processor is configured to
cause a storage device to store user attribute information, weather forecast information acquired by an environmental information acquisition unit, cooking procedure information, and distribution product information as training data for a machine learning processing system,
cause the machine learning processing system, including a generative AI model, to learn patterns from the user attribute information, the weather forecast information, the cooking procedure information, and the distribution product information,
receive user input information from a terminal device, the user input information including at least one of a free-form natural language query, a budget condition, a health condition, a nutritional condition, a cooking skill level, and a meal preparation time,
integrate the user input information with the user attribute information, the weather forecast information, the cooking procedure information, and the distribution product information, and generate a prompt sentence to be input to the generative AI model, the prompt sentence including constraints and instructions for generating at least one meal menu, ingredient set, and cooking procedure,
analyze an emotion state of a user by using an emotion estimation unit based on at least one of the user input information, voice information, and image information supplied from the terminal device, dynamically modify a content of the prompt sentence in response to the emotion state of the user and the weather forecast information so as to cause the generative AI model to preferentially generate a meal menu having at least one of a soothing characteristic and a celebratory characteristic corresponding to the emotion state and an environmental condition, input the prompt sentence into the generative AI model and obtain, as a natural language generation result, a generated text including at least one of a meal menu candidate, an ingredient candidate, a cooking procedure, and an explanation,
parse the generated text to extract structured information including the meal menu candidate, the ingredient candidate, and the cooking procedure,
map each ingredient included in the ingredient candidate to one or more purchasable products by referencing the distribution product information, calculate cost information and required time information for each meal menu candidate, and generate purchase candidate information including a list of purchasable products associated with each meal menu candidate,
transmit to the terminal device presentation information including the meal menu candidate, the associated purchase candidate information, and the cooking procedure so that the terminal device displays the meal menu candidate and enables user selection,
receive selection information from the terminal device indicating at least one selected meal menu candidate or at least one selected product, and generate order data for a delivery service based on the selection information and the purchase candidate information,
transmit the order data to the delivery service to instruct execution of an order process so that immediate delivery of food or ingredients associated with the selected meal menu candidate is performed, and
store in the storage device history information including at least a selection history, a preference history, and the emotion state of the user, and update at least one of parameters of the machine learning processing system and rules for generating the prompt sentence based on the history information to improve recommendation performance in a subsequent interaction.

### (Supplementary 2)

The system according to supplementary 1,
wherein the processor is configured to take into account expense constraint information, health state information, nutritional index information, ingredient seasonality information, cooking skill level information, and meal preparation time information of the user, convert the information into numerical and categorical conditions, append the conditions to the prompt sentence as explicit constraints, and cause the generative AI model to generate, in response to the prompt sentence, an optimized meal menu including cooking procedure information and ingredient information that satisfy the conditions.

### (Supplementary 3)

The system according to supplementary 1,
wherein the processor is configured to receive, from the terminal device, a free-form question related to food or nutrition, generate a prompt sentence including at least the question, the user attribute information, the weather forecast information, and the emotion state of the user, and cause the generative AI model, in response to the prompt sentence, to generate a natural language answer to the question and one or more related meal menu candidates.

What is claimed is:

1. A system comprising:
circuitry configured to:
receive, via a communication interface coupled to a packet-switched network, a set of attribute data and a set of contextual sensor data associated with a user from a terminal device;
construct, based on the set of attribute data, the set of contextual sensor data, a first corpus of procedural data records, and a second corpus of item availability data records stored in a non-transitory storage medium, a natural-language prompt sentence that encodes a plurality of constraint parameters derived from the set of attribute data and the set of contextual sensor data;
supply the natural-language prompt sentence to a transformer-based generative neural network model and acquire, from the transformer-based generative neural network model, inference output data comprising a structured set of candidate proposals and associated component identifiers;
parse the inference output data to extract the structured set of candidate proposals and the associated component identifiers, and map each of the associated component identifiers to one or more corresponding entries in the second corpus of item availability data records to generate a set of fulfillment data objects; and
transmit, via the communication interface coupled to the packet-switched network, the structured set of candidate proposals and the set of fulfillment data objects to the terminal device for rendering on a display of the terminal device.

2. The system according to claim 1, wherein the circuitry is further configured to:
train the transformer-based generative neural network model using a training dataset comprising the set of attribute data, the set of contextual sensor data, the first corpus of procedural data records, and the second corpus of item availability data records by executing a gradient-based optimization algorithm that minimizes a cross-entropy loss function computed over predicted output tokens and ground-truth output tokens.

3. The system according to claim 2, wherein the set of contextual sensor data comprises environmental condition data acquired from an external information service via the communication interface, the environmental condition data including at least a temperature value, a humidity value, and a precipitation indicator associated with a geographic location of the user.

4. The system according to claim 3, wherein the circuitry is further configured to:
apply an emotion estimation neural network classifier to at least one of text data, audio feature data, and image feature data received from the terminal device to compute a probability distribution over a set of emotion categories; and
select a dominant emotion category from the set of emotion categories based on the probability distribution.

5. The system according to claim 4, wherein the circuitry is further configured to:
dynamically modify a content of the natural-language prompt sentence in response to the dominant emotion category and the environmental condition data so as to cause the transformer-based generative neural network model to preferentially generate candidate proposals having at least one of a soothing characteristic and a celebratory characteristic corresponding to the dominant emotion category and an environmental condition indicated by the environmental condition data.

6. The system according to claim 5, wherein the emotion estimation neural network classifier comprises a bidirectional recurrent neural network or a transformer encoder model that receives word embeddings, mel-frequency cepstral coefficient features, or facial landmark coordinate features and outputs the probability distribution via a softmax output layer.

7. The system according to claim 1, wherein the circuitry is further configured to:
receive, from the terminal device via the communication interface, selection information indicating at least one selected candidate proposal among the structured set of candidate proposals; and
generate, based on the selection information and the associated component identifiers, a second natural-language prompt sentence incorporating a selection history, and supply the second natural-language prompt sentence to the transformer-based generative neural network model to acquire refined inference output data.

8. The system according to claim 7, wherein the circuitry is further configured to:
store, in the non-transitory storage medium, history information comprising at least a presentation history indicating which candidate proposals were presented to the user, a selection history indicating which candidate proposals were selected or rejected, and an associated emotion state detected at a time of each selection.

9. The system according to claim 8, wherein the circuitry is further configured to:
estimate, based on the history information, at least one of a user preference tendency and a user emotional state pattern by computing acceptance ratios per data category, correlation values between contextual sensor data values and selected candidate proposals, and time-of-day selection patterns; and
update at least one of parameters of the transformer-based generative neural network model and rules for constructing the natural-language prompt sentence based on the estimated user preference tendency or user emotional state pattern.

10. The system according to claim 9, wherein the first corpus of procedural data records comprises cooking procedure information including recipe identifiers, ingredient lists, step-by-step preparation instructions, estimated preparation times, difficulty level indicators, and nutritional component information, and
wherein the second corpus of item availability data records comprises product information including product identifiers, product names, unit prices, inventory availability indicators, supplier identifiers, and delivery condition parameters.

11. The system according to claim 10, wherein mapping each of the associated component identifiers to one or more corresponding entries in the second corpus of item availability data records comprises:
normalizing ingredient name strings extracted from the inference output data using a controlled vocabulary dictionary;
applying a fuzzy string matching algorithm or a synonym dictionary lookup to align normalized ingredient name strings with product name entries in the second corpus; and
filtering matched product entries based on inventory availability indicators and unit price constraints derived from the set of attribute data.

12. The system according to claim 11, wherein the set of fulfillment data objects comprises, for each component identifier, at least one matched product entry including a product identifier, a unit price, a sales location identifier, an online purchase link, and an estimated delivery time, and
wherein the circuitry is further configured to calculate an aggregated cost value for each candidate proposal by summing unit prices of matched product entries proportionally to required quantities indicated in the inference output data.

13. The system according to claim 12, wherein the circuitry is further configured to:
receive, from the terminal device, order confirmation information indicating at least a subset of matched product entries selected by the user;
generate order data comprising selected product identifiers, quantities, a delivery address, and payment parameters; and
transmit the order data, via the communication interface coupled to the packet-switched network, to an external delivery service to initiate a purchase process and a delivery process for the subset of matched product entries.

14. The system according to claim 1, wherein the circuitry is further configured to:
parse the inference output data by detecting labeled section headers and delimiter patterns within the inference output data to segment the inference output data into a plurality of proposal entries, and extract, for each proposal entry, a proposal title, a component list with quantities, and a sequence of procedural steps.

15. The system according to claim 14, wherein the circuitry is further configured to:
execute a constraint verification routine that computes nutritional index values for each proposal entry by summing component-level nutritional contributions based on quantities extracted from the inference output data; and
exclude or reduce a ranking of any proposal entry for which the computed nutritional index values exceed a threshold derived from health condition parameters included in the set of attribute data.

16. The system according to claim 15, wherein the set of attribute data comprises at least a budget constraint value, a health condition indicator, a dietary restriction flag, a skill level indicator, a temporal constraint value indicating a maximum allowable preparation time, and a preference vector encoding user preference categories.

17. The system according to claim 16, wherein the natural-language prompt sentence is constructed using a template-based prompt construction module that inserts the budget constraint value, the health condition indicator, the dietary restriction flag, the skill level indicator, the temporal constraint value, the preference vector, and a weather category derived from the set of contextual sensor data into predetermined template segments with explicit constraint labels and output format instructions.

18. A system comprising:
circuitry configured to:
receive, via a communication interface coupled to a packet-switched network, a set of attribute data comprising at least a budget constraint value, a health condition indicator, a dietary restriction flag, a skill level indicator, and a temporal constraint value, and a set of contextual sensor data comprising at least a temperature value and a weather category, from a terminal device;
construct, using a template-based prompt construction module that inserts constraint parameters into predetermined template segments, a natural-language prompt sentence based on the set of attribute data, the set of contextual sensor data, a first corpus of procedural data records stored in a relational database, and a second corpus of item availability data records stored in the relational database;
apply an emotion estimation neural network classifier to at least one of text data, audio feature data, and image feature data received from the terminal device to compute a dominant emotion category, and dynamically modify the natural-language prompt sentence based on the dominant emotion category and the weather category;
supply the modified natural-language prompt sentence to a transformer-based generative neural network model comprising a stack of self-attention layers and feed-forward layers, and acquire inference output data comprising a structured set of candidate proposals, associated component identifiers, and procedural step sequences;
parse the inference output data to extract the structured set of candidate proposals and the associated component identifiers, map each of the associated component identifiers to corresponding entries in the second corpus of item availability data records using a fuzzy string matching algorithm, and generate a set of fulfillment data objects comprising matched product entries with unit prices, inventory availability indicators, and delivery condition parameters; and
transmit the structured set of candidate proposals and the set of fulfillment data objects to the terminal device via the communication interface coupled to the packet-switched network.

19. The system according to claim 18, wherein the circuitry is further configured to:
receive order confirmation information from the terminal device indicating selected product entries, generate order data comprising product identifiers, quantities, a delivery address, and payment parameters, and transmit the order data to an external delivery service via the communication interface to initiate a purchase and delivery process.

20. A method comprising:
receiving, by circuitry via a communication interface coupled to a packet-switched network, a set of attribute data and a set of contextual sensor data associated with a user from a terminal device;
constructing, by the circuitry, based on the set of attribute data, the set of contextual sensor data, a first corpus of procedural data records, and a second corpus of item availability data records stored in a non-transitory storage medium, a natural-language prompt sentence that encodes a plurality of constraint parameters derived from the set of attribute data and the set of contextual sensor data;
supplying, by the circuitry, the natural-language prompt sentence to a transformer-based generative neural network model and acquiring, from the transformer-based generative neural network model, inference output data comprising a structured set of candidate proposals and associated component identifiers;
parsing, by the circuitry, the inference output data to extract the structured set of candidate proposals and the associated component identifiers, and mapping each of the associated component identifiers to one or more corresponding entries in the second corpus of item availability data records to generate a set of fulfillment data objects; and
transmitting, by the circuitry, via the communication interface coupled to the packet-switched network, the structured set of candidate proposals and the set of fulfillment data objects to the terminal device for rendering on a display of the terminal device.
